(19) United States
(12) Patent Application Publication
(10) Pub. No.: US 2024/0173571 A1
(43) Pub. Date: May 30, 2024

(54) SYSTEMS AND METHODS FOR DOSE VERIFICATION

(71) Applicant: SHANGHAI UNITED IMAGING HEALTHCARE CO., LTD., Shanghai (CN)

(72) Inventors: Yanfang LIU, Shanghai (CN); Cheng NI, Shanghai (CN); Fei ZHAO, Shanghai (CN); Libin WEN, Shanghai (CN); Zhao JIN, Shanghai (CN)

(73) Assignee: SHANGHAI UNITED IMAGING HEALTHCARE CO., LTD., Shanghai (CN)

(21) Appl. No.: 18/430,643

(22) Filed: Feb. 1, 2024

Related U.S. Application Data
(63) Continuation-in-part of application No. 17/823,500, filed on Aug. 30, 2022.

(30) Foreign Application Priority Data
Aug. 30, 2021 (CN) 202111006830.1
Feb. 1, 2023 (CN) 202310097112.2
May 29, 2023 (CN) 202310616337.4

(51) Int. Cl. A61N 5/10

(52) U.S. Cl. CPC A61N 5/1071; A61N 5/1039; A61N 2005/1034; A61N 2005/1074

(57) ABSTRACT

The embodiments of the present disclosure provide a method for dose verification. The method may include obtaining a predicted radiation auxiliary image of a target object at a target radiation time point; determining a target dose strategy based on the predicted radiation auxiliary image; performing, based on the target dose strategy, treatment in a current radiation fraction on the target object.

### CROSS-REFERENCE TO RELATED APPLICATIONS

This application is a continuation in part of U.S. application Ser. No. 17/823,500, filed on Aug. 30, 2022, which claims priority to Chinese Patent Application No. 202111006830.1, filed on Aug. 30, 2021, Chinese Patent Application No. 202310097112.2, filed on Feb. 1, 2023, and Chinese Patent Application No. 202310616337.4, filed on May 29, 2023, the contents of each of which are hereby incorporated by reference.

### TECHNICAL FIELD

The present disclosure relates to a medical radiation field, in particular, relates to systems and methods for dose verification.

### BACKGROUND

A radiation dose distribution may be calculated during treatment, and a patient may be subjected to radiotherapy according to a determined radiation dose. The accuracy of the calculated radiation dose distribution may affect the evaluation of radiation effect, so the calculation of the radiation dose distribution is particularly important. Therefore, it is desirable to provide systems and methods for accurately determining a radiation dose that may be used to reconstruct an actual three-dimensional radiation dose received by the patient during the treatment.

Additionally, planning quality assurance (QA) is an important part of radiation therapy that assesses effectiveness of executing a plan by comparing a planning theoretical dose of radiation treatment with an actual measured dose.

Traditionally, during treatment in a radiation fraction, an electronic portal imaging device (EPID) is used to measure an X-ray transmission image of a patient, to obtain an actual EPID image. The actual EPID image is configured to characterize an image obtained after the X-ray is transmitted to the patient and imaging on the EPID during treatment in a radiation fraction. Then, a dose error of the treatment in the radiation fraction corresponding to the actual EPID image is determined by comparing the actual EPID image with a planning EPID image calculated based on a planning computed tomography (CT) image. Therefore, some embodiments of the present disclosure provide a method, a system, and an apparatus for dose verification.

### SUMMARY

One or more embodiments of the present disclosure provide a method for dose verification implemented on a computing device having one or more processors and one or more storage devices. The method may include obtaining a predicted radiation auxiliary image of a target object at a target radiation time point; determining a target dose strategy based on the predicted radiation auxiliary image; performing, based on the target dose strategy, treatment in a current radiation fraction on the target object, obtaining a radiation auxiliary image of the target object at the target radiation time point; and reconstructing, based on the radiation auxiliary image, a radiation dose at the target radiation time point.

One or more embodiments of the present disclosure provide a a system for dose verification. The system may include at least one storage device storing a set of instructions and at least one processor in communication with the storage device. When executing the set of instructions, the at least one processor may be configured to cause the system to perform operations. The operations may include obtaining a predicted radiation auxiliary image of a target object at a target radiation time point; determining a target dose strategy based on the predicted radiation auxiliary image; performing, based on the target dose strategy, treatment in a current radiation fraction on the target object, obtaining a radiation auxiliary image of the target object at the target radiation time point; and reconstructing, based on the radiation auxiliary image, a radiation dose at the target radiation time point.

One or more embodiments of the present disclosure provide a non-transitory computer readable medium storing instructions, the instructions, when executed by at least one processor, causing the at least one processor to implement a method. The method may include obtaining a predicted radiation auxiliary image of a target object at a target radiation time point; determining a target dose strategy based on the predicted radiation auxiliary image; performing, based on the target dose strategy, treatment in a current radiation fraction on the target object, obtaining a radiation auxiliary image of the target object at the target radiation time point; and reconstructing, based on the radiation auxiliary image, a radiation dose at the target radiation time point.

One or more embodiments of the present disclosure provide a method for online radiation dose reconstruction implemented on a computing device having one or more processors and one or more storage devices. The method may include obtaining in real-time a radiation auxiliary image corresponding to each current radiation field in a plurality of radiation fields in a current treatment process for a target object; reconstructing a radiation dose in real-time based on the radiation auxiliary image corresponding to the current radiation field; and displaying in real-time the radiation dose corresponding to the current radiation field in the current treatment process, or displaying in real-time a cumulative result of radiation doses corresponding to the plurality of radiation fields in the current treatment process.

One or more embodiments of the present disclosure provide a system for online radiation dose reconstruction. The system may include at least one storage device storing a set of instructions, and at least one processor in communication with the storage device. When executing the set of instructions, the at least one processor may be configured to cause the system to perform operations. The operations may include obtaining in real-time a radiation auxiliary image corresponding to each current radiation field in a plurality of radiation fields in a current treatment process for a target object; reconstructing a radiation dose in real-time based on the radiation auxiliary image corresponding to the current radiation field; and displaying in real -time the radiation dose corresponding to the current radiation field in the current treatment process, or displaying in real-time a cumulative result of radiation doses corresponding to the plurality of radiation fields in the current treatment process.

One or more embodiments of the present disclosure provide a non-transitory computer readable medium storing instructions, the instructions, when executed by at least one processor, causing the at least one processor to implement a method. The method may include obtaining in real-time a radiation auxiliary image corresponding to each current radiation field in a plurality of radiation fields in a current treatment process for a target object; reconstructing a radiation dose in real-time based on the radiation auxiliary image corresponding to the current radiation field; and displaying in real-time the radiation dose corresponding to the current radiation field in the current treatment process, or displaying in real-time a cumulative result of radiation doses corresponding to the plurality of radiation fields in the current treatment process.

One or more embodiments of the present disclosure provide a method for determining a type of a dose error implemented on a computing device having one or more processors and one or more storage devices. The method may include obtaining one or more predicted radiation auxiliary images of a target object at a target radiation time point; obtaining a radiation auxiliary image of the target object at the target radiation time point; and determining the type of the dose error at the target radiation time point based on the first predicted radiation auxiliary image, the second predicted radiation auxiliary image, and the radiation auxiliary image. The predicted radiation auxiliary image may include a first predicted radiation auxiliary image and a second predicted radiation auxiliary image. The first predicted radiation auxiliary image may be obtained based on an initial medical scanning image of the target object. The second predicted radiation auxiliary image may be obtained based on a medical scanning image of the target object at the target radiation time point.

One or more embodiments of the present disclosure provide a system for determining a type of a dose error. The system may include at least one storage device storing a set of instructions and at least one processor in communication with the storage device. When executing the set of instructions, the at least one processor may be configured to cause the system to perform operations. The operations may include obtaining one or more predicted radiation auxiliary images of a target object at a target radiation time point; obtaining a radiation auxiliary image of the target object at the target radiation time point; and determining the type of the dose error at the target radiation time point based on the first predicted radiation auxiliary image, the second predicted radiation auxiliary image, and the radiation auxiliary image. The predicted radiation auxiliary image may include a first predicted radiation auxiliary image and a second predicted radiation auxiliary image. The first predicted radiation auxiliary image may be obtained based on an initial medical scanning image of the target object. The second predicted radiation auxiliary image may be obtained based on a medical scanning image of the target object at the target radiation time point.

One or more embodiments of the present disclosure provide a non-transitory computer readable medium storing instructions, the instructions, when executed by at least one processor, causing the at least one processor to implement a method. The method may include obtaining one or more predicted radiation auxiliary images of a target object at a target radiation time point; obtaining a radiation auxiliary image of the target object at the target radiation time point; and determining the type of the dose error at the target radiation time point based on the first predicted radiation auxiliary image, the second predicted radiation auxiliary image, and the radiation auxiliary image. The predicted radiation auxiliary image may include a first predicted radiation auxiliary image and a second predicted radiation auxiliary image. The first predicted radiation auxiliary image may be obtained based on an initial medical scanning image of the target object. The second predicted radiation auxiliary image may be obtained based on a medical scanning image of the target object at the target radiation time point.

### BRIEF DESCRIPTION OF THE DRAWINGS

The present disclosure is further illustrated in terms of exemplary embodiments, and these exemplary embodiments are described in detail with reference to the drawings. These embodiments are not restrictive. In these embodiments, the same number indicates the same structure, wherein:

FIG. 1 is a schematic diagram illustrating an exemplary application scenario of a system for dose verification according to some embodiments of the present disclosure;

FIG. 2 is a flowchart illustrating an exemplary process for dose verification according to some embodiments of the present disclosure;

FIG. 3 is a flowchart illustrating an exemplary process for determining a target dose strategy according to some embodiments of the present disclosure;

FIG. 4 is a flowchart illustrating an exemplary process for obtaining a predicted radiation auxiliary image according to some embodiments of the present disclosure;

FIG. 5 is a block diagram illustrating an exemplary system for determining a radiation dose according to some embodiments of the present disclosure;

FIG. 6 is a flowchart illustrating an exemplary process for determining a radiation dose according to some embodiments of the present disclosure;

FIG. 7 is a flowchart illustrating an exemplary current iteration of one or more iterations according to some embodiments of the present disclosure;

FIG. 8 is a flowchart illustrating another exemplary current iteration of the one or more iterations according to some embodiments of the present disclosure;

FIG. 9 is a flowchart illustrating an exemplary process for obtaining a target scanning image of a target object according to some embodiments of the present disclosure;

FIG. 10 is a flowchart illustrating an exemplary process for determining a fluence map according to some embodiments of the present disclosure;

FIG. 11 is a flowchart illustrating an exemplary process for determining a radiation dose according to some embodiments of the present disclosure;

FIG. 12 is a flowchart illustrating an exemplary process for online radiation dose reconstruction according to some embodiments of the present disclosure;

FIG. 13 is an exemplary schematic diagram illustrating displaying in real-time a reconstruction result according to some embodiments of the present disclosure;

FIG. 14 is a flowchart illustrating an exemplary process for determining an evaluation result according to some embodiments of the present disclosure;

FIG. 15 is a flowchart illustrating an exemplary process for determining a type of dose error according to some embodiments of the present disclosure;

FIG. 16 is a flowchart illustrating an exemplary process for determining a first type of dose error according to some embodiments of the present disclosure;

FIG. 17 is a flowchart illustrating an exemplary process for determining a second type of dose error according to some embodiments of the present disclosure;

FIG. 18 is a flowchart illustrating an exemplary process for performing treatment in a radiation fraction according to some embodiments of the present disclosure;

FIG. 19 is a flowchart illustrating an exemplary process for determining a second predicted radiation auxiliary image according to some embodiments of the present disclosure;

FIG. 20 is a flowchart illustrating an exemplary process for determining a dose analysis result according to some embodiments of the present disclosure;

FIG. 21 is a flowchart illustrating an exemplary process for determining a type of dose error according to some embodiments of the present disclosure;

FIG. 22 is a schematic diagram illustrating determining a first type of dose error according to some embodiments of the present disclosure;

FIG. 23 is a schematic diagram illustrating determining a second type of dose error according to some embodiments of the present disclosure;

FIG. 24 is a schematic diagram illustrating dose analysis of treatment in a radiation fraction according to some embodiments of the present disclosure;

FIG. 25 is an exemplary diagram illustrating modules of a dose verification system according to some embodiments of the present disclosure;

FIG. 26 is an exemplary diagram illustrating modules of a system for online radiation dose reconstruction according to some embodiments of the present disclosure;

FIG. 27 is an exemplary diagram illustrating modules of a system for determining a type of dose error according to some embodiments of the present disclosure;

FIG. 28 is a schematic diagram illustrating an exemplary hardware and/or software components of a computing device according to some embodiments of the present disclosure;

FIG. 29 is a flowchart illustrating an exemplary process for determining a dose error according to some embodiments of the present disclosure; and

FIG. 30 is a flowchart illustrating an exemplary process of a method for dose error evaluation according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the accompanying drawing in the following description is merely some examples or embodiments of the present disclosure, for those skilled in the art, the present disclosure may further be applied in other similar situations according to the drawings without any creative effort. Unless obviously obtained from the context or the context illustrates otherwise, the same numeral in the drawings refers to the same structure or operation.

It will be understood that the term “system,” “device,” “unit,” and/or “module” used herein are one method to distinguish different assemblies, elements, parts, sections or assemblies of different levels in ascending order. However, if other words may achieve the same purpose, the words may be replaced by other expressions.

As used in the disclosure and the appended claims, the singular forms “a,” “an,” and “the” include plural referents unless the content clearly dictates otherwise. Generally speaking, the terms “comprise” and “include” only imply that the clearly identified steps and elements are included, and these steps and elements may not constitute an exclusive list, and the method or device may further include other steps or elements.

The flowcharts used in the present disclosure illustrate operations that the system implements according to the embodiment of the present disclosure. It should be understood that a previous operation or a subsequent operation of the flowcharts may not be accurately implemented in order. Instead, a plurality of steps may be processed in reverse or simultaneously. Moreover, other operations may further be added to these procedures, or one or more steps may be removed from these procedures.

The present disclosure describes a system for determining a radiation dose, the system may iteratively calculate an output flux of an accelerator based on a measured electronic portal imaging device (EPID) image and related parameters of a radioactive source, and may accurately reconstruct an actual three-dimensional (3D) dose received by a patient during treatment, and thus, a calculation model for determining the radiation dose may be greatly simplified and the calculation accuracy may be improved.

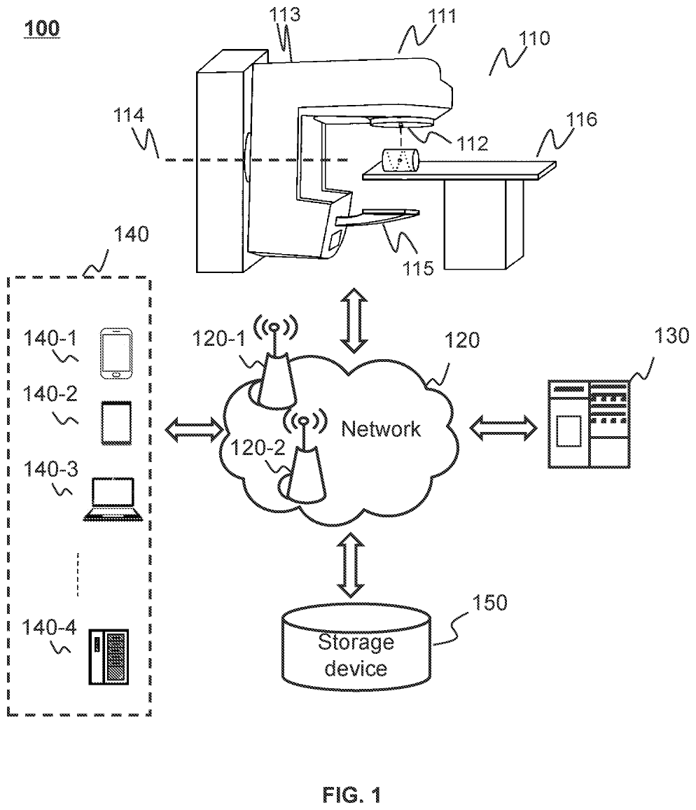

FIG. 1 is a schematic diagram illustrating an exemplary application scenario of a system for dose verification according to some embodiments of the present disclosure.

In some embodiments, a system 100 for dose verification may be applied to a medical system platform. For example, the system 100 may determine the radiation dose received by a target object (e.g., a patient) at a target radiation time point. As another example, the system 100 may determine the radiation dose received by the target object (e.g., a patient) through an obtained auxiliary image at the target radiation time point. As shown in FIG. 1, the system 100 may include a radiation device 110, a network 120, a processing device 130, a terminal 140, and a storage device 150. The various assemblies in the system 100 may be connected with each other through the network 120. For example, the processing device 130 and the radiation device 110 may be connected or communicated through the network 120.

The radiation device 110 may transmit one or more radiation beams to the target object (e.g., a patient or a phantom). In some embodiments, the radiation device 110 may include a linear accelerator 111 (also may be referred to as linac). The linear accelerator 111 may generate and emit the radiation beam(s) (e.g., an X-ray beam) from a treatment head 112. The radiation beam(s) may pass through one or more collimators with a specific shape (e.g., a multi-leaf collimator) and be transmitted to the target object. In some embodiments, the radiation beam(s) may include electrons, photons, or any other types of radiation. In some embodiments, energy of the radiation beam(s) may be in a megavolt level (i.e., >1 MeV), and the radiation beam(s) may also be referred to as megavolt radiation beam(s). The treatment head 112 may be coupled to a gantry 113. The gantry 113 may rotate, for example, clockwise or counterclockwise around a gantry rotation axis 114. The treatment head 112 may rotate with the gantry 113 together. In some embodiments, the radiation device 110 may include an imaging assembly 115. The imaging assembly 115 may receive the radiation beam(s) passing through the target object and acquire projection image(s) of the patient or the phantom before, during, and/or after the radiation or correction process. The imaging assembly 115 may include an analog detector, a digital detector, or any combination thereof. The imaging assembly 115 may be attached to the gantry 113 in any manner, and/or include a retractable housing. Therefore, when the gantry 113 rotates, the treatment head 112 and the imaging assembly 115 may rotate synchronously. In some embodiments, the imaging assembly 115 may include an EPID. In some embodiments, the radiation device 110 may also include a bed 116. The bed 116 may support the patient during the radiation or imaging, and/or support the phantom during the correction process of the radiation device 110. The bed 116 may be adjusted according to different application scenarios.

The network 120 may include any suitable network capable of facilitating the exchange of information and/or data of the system 100. The information and/or data may include one or more radiation auxiliary images transmitted from the radiation device 110 to the processing device 130. For example, the processing device 130 may obtain the radiation auxiliary image (e.g., an EPID image) determined by the imaging assembly 115 from the radiation device 110 via the network 120. As another example, the processing device may obtain user (e.g., a doctor) instruction from the terminal 140 via the network 120. In some embodiments, the network 120 may be any type of wired or wireless network. For example, the network 120 may include cable network, wired network, optical fiber network, telecommunication network, internal network, Internet, area network (LAN), wide area network (WAN), wireless area network (WLAN), metropolitan area network (man), public switched telephone network (PSTN), Bluetooth network, ZigBee network, near field communication (NFC) network, ultra-wideband (UWB) network, mobile communication (1g, 2G, 3G, 4G, 5g) network, narrowband Internet of things (NB IoT), infrared communication network, or any combination thereof. In some embodiments, the network 120 may include one or more network access points. For example, the network 120 may include wired or wireless network access points, such as base stations and/or internet switching points 120-1, 120-2, . . . , through these access points, one or more assemblies of the system 100 may be connected with the network 120 to exchange the data and/or information.

The terminal 140 may communicate and/or be connected with the radiation device 110, the processing device 130, and/or the storage device 150. For example, the terminal 140 may determine a dose determination result during the radiotherapy from the processing device 130. As another example, the terminal 140 may obtain an image (e.g., a radiation auxiliary image) acquired by the radiation device 110., and transmit the image to the processing device 130 for processing. In some embodiments, the terminal 140 may include a mobile device 140-1, a tablet computer 140-2, a laptop computer 140-3, a desktop computer 140-4, or any combination thereof. For example, the mobile device 140-1 may include a mobile phone, a personal digital assistant (PDA), a game device, a navigation device, or any combination thereof. In some embodiments, the terminal 140 may include an input device, an output device, or the like. The input device may include alphanumeric and other keys. The input device may choose a keyboard input, a touch screen (e.g., with tactile or tactile feedback) input, a voice input, an eye-tracking input, a brain monitoring system input, or any other similar input mechanism. Input information received by the input device may be transmitted to the processing device 130 via a bus for further processing. The other types of input devices may include a cursor control device, such as a mouse, a trackball, a cursor direction key, or the like. The output device may include a display, a speaker, a printer, or any combination thereof. In some embodiments, the terminal 140 may be part of the processing device 130. In some embodiments, the terminal 140 and the processing device 130 may be integrated as a control device of the radiation device 110, such as an operation console. In some embodiments, the terminal 140 may be omitted.

The storage device 150 may store data, instructions, and/or any other information. In some embodiments, the storage device 150 may store information for behaviors of a user to control the radiation device 110. In some embodiments, the storage device 150 may store data obtained from the radiation device 110, the terminal 140, and/or the processing device 130. In some embodiments, the storage device 150 may store data and/or instructions used by the processing device 130 to perform or use to accomplish the example methods described in the present disclosure. In some embodiments, the storage device 150 may include a mass storage, a removable memory, a volatile read-write memory, a read-only memory (ROM), or any combination thereof. The exemplary mass storage may include a magnetic disk, an optical disk, a solid state disk, or the like. The exemplary removable storage may include a flash drive, a floppy disk, an optical disk, a memory card, a compact disk, a magnetic tape, or the like. The exemplary volatile read-write memory may include a random access memory (RAM). The exemplary RAM may include a Dynamic Random Access Memory (DRAM), a Double Data Rate Synchronous Dynamic Random Access Memory (DDRSDRAM), a Static Random Access Memory (SRAM), a Thyristor Random Access Memory (T-RAM), and a Zero Capacitance Random Access Memory (Z-RAM), etc. The exemplary read-only memory may include a masked read-only memory (MROM), programmable read-only memory (PROM), an erasable programmable read-only memory (EPROM), an electrically erasable programmable read-only memory (EEPROM), an optical disk read-only memory (CD-ROM), a digital multifunctional disk read-only memory, or the like. In some embodiments, the storage device 150 may be implemented on a cloud platform.

In some embodiments, the storage device 150 may be connected with the network 120 to communicate with at least one other assembly (e.g., the processing device 130, the terminal 140) in the system 100. At least one assembly of the system 100 may access data or instructions stored in the storage device 150 via the network 120. In some embodiments, the storage device 150 may be part of the processing device 130.

In some embodiments, the system 100 may also include one or more power supplies (not shown in FIG. 1) connected with one or more assemblies of the system 100 (e.g., the processing device 130, the radiation device 110, the terminal 140, the storage device 150, etc.).

It should be noted that the above description is provided for illustrative purposes only and is not intended to limit the scope of the present disclosure. For those skilled in the art, many changes and modifications can be made under the guidance of the content of the present disclosure. The features, structures, methods, and other features of the exemplary embodiments described herein may be combined in various ways to obtain additional and/or alternative exemplary embodiments. For example, the storage device 150 may be a data storage device including a cloud computing platform, such as a public cloud, a private cloud, a community, a hybrid cloud, and the like. However, these changes and modifications may not deviate from the scope of the present disclosure.

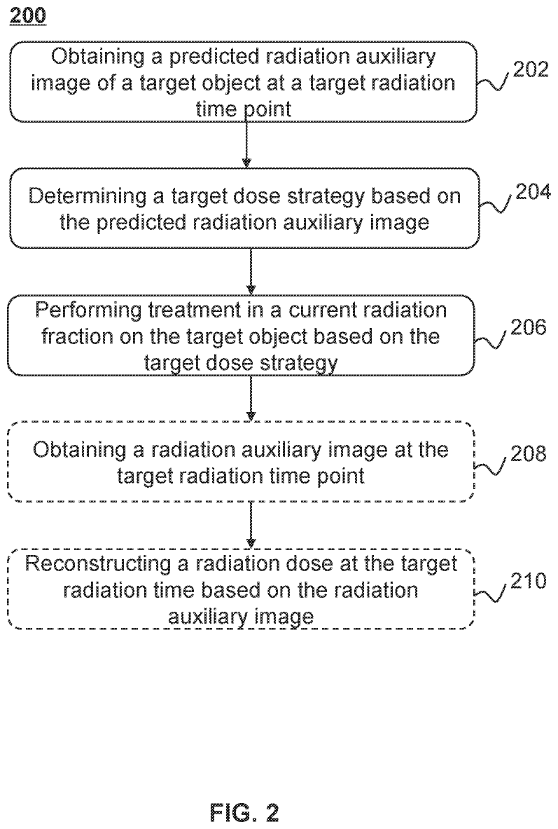

FIG. 2 is a flowchart illustrating an exemplary process for dose verification according to some embodiments of the present disclosure. As shown in FIG. 2, process 200 includes one or more of the following operations. In some embodiments, process 200 may be performed by a processing device or a system for dose verification.

In 202, a predicted radiation auxiliary image of a target object at a target radiation time point may be obtained.

The target object may include a patient or any other medical object (e.g., an animal such as a test mouse), etc. In some embodiments, the target object may be a part of the patient or any other medical object, including an organ or tissue, such as a heart, a lung, a rib, an abdominal cavity, etc.

A radiation treatment plan (TPS) may be determined before radiation therapy for the target object (e.g., a cancer patient, a cancerous organ or tissue of the cancer patient, etc.). The radiation treatment plan may include detailed operations of a radiation device (e.g., the radiation device 110) throughout the radiation treatment. For example, the radiation treatment plan may specify a plurality of nodes (also referred to as control nodes), and each of the control nodes may correspond to a time point. The radiation treatment plan may indicate, a rotation angle of the gantry 113, moving positions of leaves of a multi-leaf collimator and/or tungsten gate, a dose of rays emitted by the linear accelerator 111, etc., at each time point. A radiation ray may be emitted at the control nodes (e.g., in a static intensity modulated radiation) or may be emitted continuously between two control nodes (e.g., in a dynamic intensity modulated radiation). Therefore, a target radiation time point may be a time point corresponding to a control node, or a time point between two control nodes. At this time point, the radiation device (e.g., the radiation device 110) may begin or stop to emit radiation rays.

The predicted radiation auxiliary image may refer to a medical image obtained based on an initial radiation treatment plan (also referred to as a treatment plan) for the target object. In some embodiments, the processing device may obtain a predicted radiation auxiliary image based on an initial radiation treatment plan and an initial medical image of the target object. An initial medical scanning image may refer to a medical scanning image of a target object used to develop an initial treatment plan for radiation treatment before the target object undergoes radiation treatment. In some embodiments, the initial medical scanning image may be a computed tomography (CT) image, a magnetic resonance (MR) image, etc.

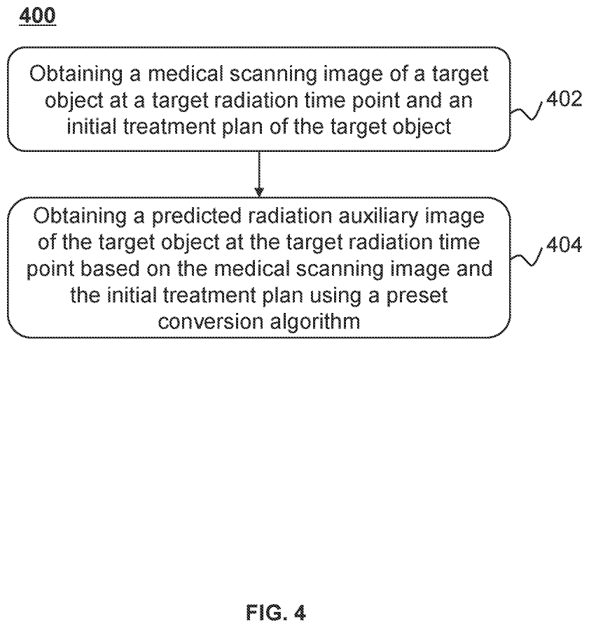

In some embodiments, after obtaining an initial treatment plan, the processing device may obtain a predicted radiation auxiliary image based on the treatment plan corresponding to the target radiation time point in the initial treatment plan and the initial medical scanning image of the target object. For example, the processing device may obtain, based on the initial medical scanning image of the target object and a radiation dose in the treatment plan, the predicted radiation auxiliary image through calculation. In some embodiments, the predicted radiation auxiliary image may include an EPID image, which may also be referred to as a first predicted radiation auxiliary image. More description of obtaining the predicted radiation auxiliary image may be found in FIG. 4 and related descriptions thereof.

In 204, a target dose strategy may be determined based on the predicted radiation auxiliary image.

The target dose strategy may refer to a determined manner or way of performing the radiation treatment on a target object. In some embodiments, the target dose strategy may include continuing treatment and/or modifying the plan. For example, if a current treatment plan is determined to be continued based on the predicted radiation auxiliary image, the target object may continue to be treated using the current treatment plan. If a radiation dose in the current treatment plan is excessively high or excessively low for the target object, the treatment plan may be modified, and the radiation treatment may be performed on the target object based on the modified treatment plan.

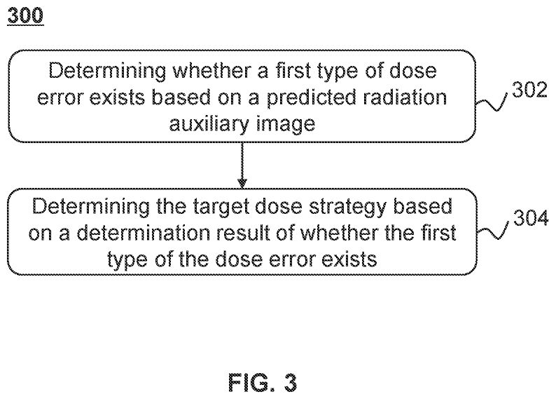

In some embodiments, the processing device may determine, based on the predicted radiation auxiliary image, whether a dose error exists in the current treatment plan. If the dose error exists, the treatment plan may be modified to determine the target dose strategy. If the dose error does not exist, the target dose strategy may be determined based on the initial treatment plan or an original treatment plan. More descriptions regarding determining the target dose strategy may be found in FIG. 3 hereinafter.

In 206, treatment in a current radiation fraction may be performed on the target object based on the target dose strategy.

According to a pre-specified treatment plan, the target object may need to receive one or more treatments. In some embodiments, a treatment process may include a plurality of radiation fractions, and a total radiation dose of the treatment process may be divided into a plurality of dose units. A portion of the total radiation dose may be delivered to the target object in a radiation fraction. In some embodiments, the processing device may determine the current treatment plan based on the target dose strategy and perform the treatment in the current radiation fraction on the target object based on the current treatment plan.

In 208, the radiation auxiliary image at the target radiation time point may be obtained.

The radiation auxiliary image may refer to a medical image obtained during the treatment in the radiation fraction. The radiation auxiliary image may include a medical image obtained by an imaging assembly of the radiation device based on data generated by received ray(s) passing through the target object during the radiation treatment. For example, the radiation auxiliary image may be a medical image received by the imaging assembly (e.g., the imaging assembly 115) and obtained based on data generated by received ray(s) that are delivered by a radiation source (e.g., the linear accelerator 111) and passes through the target object at a radiation time point. In some embodiments, the radiation auxiliary image may include an EPID (electronic portal imaging device) image. The radiation auxiliary image may also be referred to as an actual EPID image.

In 210, a radiation dose at the target radiation time point may be reconstructed based on the radiation auxiliary image.

The radiation dose may refer to an amount of radiation energy delivered to a tumor or a treatment region during the radiation treatment. The radiation dose at the target radiation time point refers to an amount of radiation energy delivered to the tumor or the treatment region from a time point of starting radiation delivery in the current radiation fraction to a current time point during the treatment.

In some embodiments, the processing device may update, based on the radiation auxiliary image, and data related to the radiation source and an initial fluence map corresponding to the target radiation time point, the initial fluence map through one or more iterations to obtain a target fluence map. In some embodiments, the processing device may reconstruct the radiation dose at the target radiation time point based on the target fluence map.

In some embodiments of the present disclosure, by obtaining in real-time the radiation auxiliary image for real-time reconstruction of the radiation dose in the current radiation fraction, the radiation dose of the radiation fraction may be provided to a doctor in real-time, and the doctor may optimize the subsequent treatment plan based on a real-time displayed radiation dose to treat the patient better. At the same time, by displaying in real-time the radiation dose, the doctor can take note of the radiation dose of the treatment in time, and the subsequent operations such as adjusting an expected dose corresponding to a subsequent radiation field of the treatment, etc., may be performed more proactively, thus realizing a real-time optimization of the treatment plan. Moreover, in the process of reconstructing the radiation dose, the target dose strategy may be determined based on the predicted radiation auxiliary image and the radiation auxiliary image obtained in the current radiation fraction, making the treatment plan in actual treatment more suitable for a current situation of the target object, which reduces the dose error of the treatment and effectively improve accuracy of treatment.

FIG. 3 is a flowchart illustrating an exemplary process for determining a target dose strategy according to some embodiments of the present disclosure. As shown in FIG. 3, process 300 may include one or more of the following operations. In some embodiments, the process 300 may be performed by a processing device or a system for dose verification.

In 302, whether a first type of dose error exists may be determined based on a predicted radiation auxiliary image.

The first type of dose error may refer to a dose error caused by variation of a target object (e.g., positioning variation and/or body posture variation of a target object). In some embodiments, the variation of the target object may include a position, posture and/or somatotype of the target object.

In some embodiments, the predicted radiation auxiliary image may include a first predicted radiation auxiliary image and/or a second predicted radiation auxiliary image. For example, the processing device may obtain a first assessment result by comparing the first predicted radiation auxiliary image and the second predicted radiation auxiliary image using a preset assessment algorithm. The processing device may determine, based on the first assessment result, whether a dose error at a target radiation time point belongs to the first type. For example, the processing device may determine, based on the first assessment result, whether a dose error exists. If the dose error exists, the processing device may determine whether the dose error belongs to the first type.

The first predicted radiation auxiliary image may be obtained based on an initial medical scanning image of the target object, and the second predicted radiation auxiliary image may be obtained based on a medical scanning image of the target object during treatment in a current radiation fraction. In some embodiments, the initial medical scanning image and the medical scanning image may be obtained at different time points. For example, the initial medical scanning image may be obtained in the treatment planning stage, while the medical scanning image may be obtained during the current radiation fraction. As another example, the initial medical scanning image may be obtained in the treatment planning stage, while the medical scanning image may be obtained during a previous radiation fraction.

In some embodiments, the preset assessment algorithm may be an algorithm for a quantitative assessment of a similarity between two images, such as an image alignment algorithm, a root-mean-square error determination, a deep learning algorithm, a gamma-pass evaluation algorithm, etc.

The first assessment result may reflect a similarity between the first predicted radiation auxiliary image and the second predicted radiation auxiliary image. In some embodiments, if the first assessment result shows that the similarity between the first predicted radiation auxiliary image and the second predicted radiation auxiliary image is less than or equal to a first similarity threshold, the dose error at the target radiation time point may be determined to belong to the first type.

In 304, the target dose strategy may be determined based on a determination result of whether the first type of dose error exists.

In some embodiments, if the current dose error belongs to the first type, the target dose strategy may be determined based on a preset error adjustment strategy. The preset error adjustment strategy may include a first error adjustment strategy corresponding to the first type of dose error and a second error adjustment strategy corresponding to a second type of dose error. If the dose error belongs to the first type of dose error, the first error adjustment strategy in the preset error adjustment strategy may be determined as the target dose strategy. The first error adjustment strategy may include whether to continue treatment, whether to modify the treatment plan, etc. Descriptions regarding the second type of dose error may be found hereinafter, which may not be repeated here.

FIG. 4 is a flowchart illustrating an exemplary process for obtaining a predicted radiation auxiliary image according to some embodiments of the present disclosure. As shown in FIG. 4, process 400 may include one or more of the following operations. In some embodiments, the process 400 may be performed by a processing device or a system for dose verification.

In 402, a medical scanning image of a target object at a current radiation fraction and an initial treatment plan of the target object may be obtained.

The medical scanning image may include an initial medical scanning image of the target object used to make an initial treatment plan and/or a medical scanning image of the target object obtained before the current radiation fraction. The initial treatment plan refers to a radiation treatment plan made for specific circumstances of the target object based on the initial medical scanning image of the target object. In some embodiments, the processing device may read the initial treatment plan of the target object obtained by pre-scanning from a storage device, a database, or an imaging device. The initial treatment plan may be predetermined and stored. In some embodiments, the processing device may obtain the medical scanning image of the target object at the current radiation fraction by scanning the target object using the imaging device.

In 404, a predicted radiation auxiliary image of the target object at the current radiation fraction may be obtained based on the medical scanning image and the initial treatment plan using a preset conversion algorithm.

The preset conversion algorithm may be used to convert the medical scanning image into a predicted radiation auxiliary image. The predicted radiation auxiliary image may be used to characterize a dose distribution of the target object under a certain radiation dose. In some embodiments, the processing device may obtain, after obtaining the medical scanning image and the initial treatment plan, the predicted radiation auxiliary image by using the preset conversion algorithm based on the medical scanning image and the initial treatment plan to perform an image conversion process. In some embodiments, the preset conversion algorithm may include an inverse projection algorithm, a filtered inverse projection algorithm, and a model-based image conversion algorithm, etc.

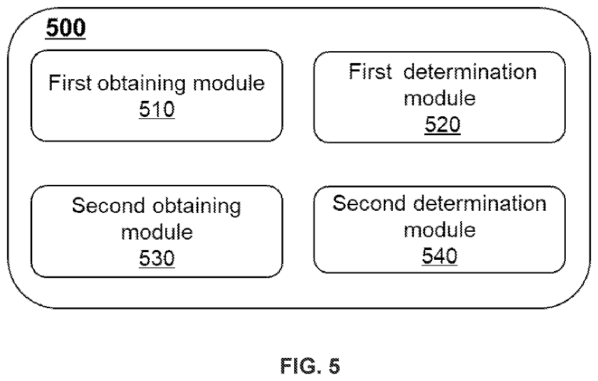

FIG. 5 is a block diagram illustrating an exemplary system for determining a radiation dose according to some embodiments of the present disclosure.

As shown in FIG. 5, the processing device 130 may include a first obtaining module 510, a first determination module 520, a second obtaining module 530, and a second determination module 540.

The first obtaining module 510 may be used to obtain data related to radiation source, a radiation auxiliary image of the target object at the target radiation time point, and an initial fluence map corresponding to the target radiation time point. The data related to radiation source may be used to describe parameters of devices and/or assemblies related to ray delivery. The devices and/or assemblies may include a radioactive source, an accelerator, a collimator, or the like. The exemplary parameters may include a radiation beam energy, a radiation beam spot size, collimator physical parameters (such as a blade length, a blade thickness, or a range of motion of a multi-leaf collimator), etc. The radiation auxiliary image may include a medical image obtained by the imaging assembly of the radiation device based on data generated by received ray(s) passing through the target object during the radiation. The radiation auxiliary image may include an EPID image. In some embodiments, the initial fluence map corresponding to the target radiation time point may be a preset image. For example, the initial fluence map may be any medical image. As another example, the initial fluence map may be an image obtained by processing the data received by the imaging assembly after radiation beam(s) emitted by the radiation device passes through the phantom.

In some embodiments, the data related to radiation source may include a source model of the radioactive source.

The first determination module 520 may be used to determine a target fluence map corresponding to the target radiation time point by one or more iterations based on the radiation auxiliary image, the initial fluence map, and the data related to radiation source. The target fluence map may reflect relevant state information of the radioactive source at the target radiation time point. The first determination module 520 may determine a final target fluence map by repeatedly simulating, for example, a physical motion process of chief ray particles. The first determination module 520 may repeatedly determine and update the fluence map in one or more iterations. A current iteration of the one or more iterations may include a simulation and a process of updating the fluence map. In some embodiments, the radiation auxiliary image may be a corrected image. For example, the first determination 520 may correct the radiation auxiliary image to obtain a corrected radiation auxiliary image, and determine a target fluence map corresponding to the target radiation time point by the one or more iterations based on the corrected radiation auxiliary image, the initial fluence map, and the data related to radiation source. In some embodiments, the correction may include bad point correction, dark current correction, gain correction, geometric correction, or any combination thereof.

In some embodiments, in the current iteration of the one or more iterations, the first determination module 520 may obtain object information of the target object, and determine a prediction image of radiation in the current iteration based on the data related to radiation source, a current fluence map corresponding to the current iteration, and the object information of the target object. The object information of the target object may include scanning image information of the target object (e.g., a positioning image of the target object, a plan image of the target object). The exemplary scanning image information may include Computed Radiography (CR) image information, Digital Radiography (DR) image information, Computed Tomography (CT) image information, Magnetic Resonance Imaging (MRI) image information, Positron Emission Computed Tomography (PET) image information, or any combination thereof. In some embodiments, the object information may be acquired in advance before the target radiation time point. In some embodiments, the first determination module 520 may determine the prediction image of radiation in the current iteration based on the data related to radiation source, the current fluence map corresponding to the current iteration, and the object information of the target object using a Monte Carlo Method.

In some embodiments, the first determination module 520 may determine whether the radiation auxiliary image and the prediction image of radiation in the current iteration satisfy a first judgment condition. The first judgment condition may include a prediction image of radiation in the current iteration being convergent to the radiation auxiliary image. The convergence may mean that a difference between the prediction image of radiation and the radiation auxiliary image in the current iteration is less than a preset threshold. The difference may be related to a difference between pixel values of the corresponding pixels in the two images. In response to the radiation auxiliary image and the prediction image of radiation in the current iteration satisfying the first judgment condition, the first determination module 520 may designate the current fluence map corresponding to the current iteration as the target fluence map. In response to the radiation auxiliary image and the prediction image of radiation in the current iteration do not satisfy the first judgment condition, the first determination module 520 may update the current fluence map corresponding to the current iteration, and designate the updated current fluence map corresponding to the current iteration as a current fluence map corresponding to a next iteration. The first determination module 520 may determine a first difference between the radiation auxiliary image and the prediction image of radiation in the current iteration. The first difference may be a first difference matrix between a first matrix representing the radiation auxiliary image and a second matrix representing the prediction image of radiation in the current iteration. The first determination module 520 may update a current fluence map corresponding to the current iteration based on the first difference.

In some embodiments, in the current iteration of the one or more iterations, the first determination module 520 may obtain the object information of the target object, and determine a prediction image of main radiation beam and a scattering ratio in the current iteration based on the data related to radiation source, the current fluence map corresponding to the current iteration, and the object information of the target object. The prediction image of main radiation beam may be an image formed by chief ray particles after removing scattering particles from the radiation beam. The scattering ratio may be a ratio between the amount of the scattering particles and the amount of the chief ray particles. In some embodiments, the first determination module 520 may determine a prediction image of main radiation beam and a scattering ratio in the current iteration based on the data related to radiation source, the current fluence map corresponding to the current iteration, and the object information of the target object using the Monte Carlo Method.

In some embodiments, the first determination module 520 may determine a descattering reference image in the current iteration based on the scattering ratio and the radiation auxiliary image and determine whether the descattering reference image and the prediction image of main radiation beam in the current iteration satisfy a second judgment condition. The descattering reference image may be determined based on the chief ray particles in an actual dose captured by a detection assembly of the radiation device. The second judgment condition may include the prediction image of main radiation beam in the current iteration being convergent to the descattering reference image in the current iteration. The convergence may mean that a difference between the prediction image of main radiation beam in the current iteration and the descattering reference image in the current iteration is less than a preset threshold. The difference may be a difference between the pixel values of the corresponding pixels in the two images. In response to the descattering reference image and the prediction image of main radiation beam in the current iteration satisfying the second judgment condition, the first determination module may designate the current fluence map corresponding to the current iteration as the target fluence map. In response to the descattering reference image and the prediction image of main radiation beam in the current iteration not satisfying the second judgment condition, the first determination module 520 may update the current fluence map corresponding to the current iteration and designate the updated current fluence map corresponding to the current iteration as a current fluence map corresponding to a next iteration. In some embodiments, the first determination module may determine a second difference between the descattering reference image and the prediction image of main radiation beam in the current iteration. The second difference may be a second difference matrix between a third matrix representing the descattering reference image in the current iteration and a fourth matrix representing the prediction image of main radiation beam in the current iteration. The first determination module 520 may update the current fluence map corresponding to the current iteration based on the second difference.

The second obtaining module 530 may be used to obtain a target scanning image of the target object. In some embodiments, the second obtaining module 530 may obtain a plurality of scanning images of the target object before the target radiation time point and determine a plurality of prediction phase images of the target object at the target radiation time point corresponding to the plurality of phases respectively. The plurality of scanning images may reflect different motion states of the target object in one or more autonomous motion cycles. The plurality of scanning images may be pre-scanned and determined before the target radiation time point. In some embodiments, the plurality of scanning images may include one or more four-dimensional computed tomography (4D-CT) images obtained based on an T imaging device or one or more online 4D-CT images. In some embodiments, a prediction phase image may refer to a prediction image reflecting a state of the target object at the target radiation time point. In order to determine the plurality of prediction phase images corresponding to the plurality of phases respectively, the second obtaining module 530 may obtain treatment planning information and determine planning delivery information at the target radiation time point based on the treatment planning information. The planning delivery information may include a radiation beam intensity, a radiation beam conformal shape, a radiation dose, or the like. For each phase of the plurality of phases, the second obtaining module 530 may obtain relevant information of the phase. The relevant information of the phase may include state information or phase information of the target object in the phase. For example, the relevant information of the phase may include stages of physiological movement (e.g., systolic phases of cardiac movement, diastolic phases of cardiac movement, etc.) of the target object (e.g., a patient, organ, or tissue of a patient), postures (such as lying down, lying on the side, etc.), a state, or a body shape of the target object, etc. The second obtaining module 530 may determine the prediction phase image corresponding to the phase based on the planning delivery information and the relevant information of the phase. For example, the second obtaining module 530 may obtain the prediction phase image using simulation.

In some embodiments, the second obtaining module 530 may determine, from the plurality of prediction phase images, a matched image that matches the radiation auxiliary image. The matched image may refer to a prediction phase image similar to the radiation auxiliary image corresponding to the target radiation time point. For example, the state of the target object displayed in the matched image may be the closest to the state of the target object displayed in the radiation auxiliary image. In some embodiments, the second obtaining module 530 may determine the matched image that matches the radiation auxiliary image using a feature matching algorithm. For example, the second obtaining module 530 may compare a feature distribution (e.g., a grayscale distribution) of each of the plurality of prediction phase images with a feature distribution (e.g., a grayscale distribution) of the radiation auxiliary image and choose the prediction phase image whose feature distribution is closest to the feature distribution of the radiation auxiliary image as the matched image. In some embodiments, the second obtaining module 530 may determine first position information of a target tissue included in the radiation auxiliary image and second position information of the target tissue included in each image of the plurality of the prediction phase images. The target tissue may refer to an identifiable tissue in the target object, such as tumor area or organ. The second obtaining module 530 may determine the matched image of the radiation auxiliary image based on the first position information and the second position information. For example, the second obtaining module 530 may compare the first position information and the second position information corresponding to each prediction phase image. If the first position information matches the second position information corresponding to a prediction phase image, the second obtaining module 530 may designate the prediction phase image as the matched image.

In some embodiments, the second obtaining module may determine a third difference of each prediction phase image in the plurality of prediction phase images and the radiation auxiliary image. The third difference may refer to a difference between a matrix representing the radiation auxiliary image and a matrix representing the prediction phase image. The second obtaining module 530 may determine a minimum value in the plurality of third differences, and designate the prediction phase image corresponding to the minimum value as the matched image.

In some embodiments, the second obtaining module 530 may determine a target phase corresponding to the matched image and designate a scanning image corresponding to the target phase as the target scanning image.

The second determination module 540 may be used to determine the radiation dose received by the target object at the target radiation time point based on the target fluence map, the target scanning image, and the data related to radiation source. The second determination module 540 may determine the radiation dose received by the target object at the target radiation time point using the Monte Carlo Method. In some embodiments, the Monte Carlo method may be used to simulate various physical processes of ray particles in the target object (e.g., scattering, attenuation, etc.). For example, the second determination module 540 may simulate a transport process of the ray particles using the Monte Carlo method, for example, under a parameter condition of devices and/or assemblies used for ray delivery as reflected by the data related to radiation source and a condition of the radioactive source reflected by the target fluence map, after rays pass through an inner region of the target object reflected by the target scanning image, a final dose distribution may be obtained. Based on the dose distribution, the second determination module 540 may determine the radiation dose received by the target object at the target radiation time point.

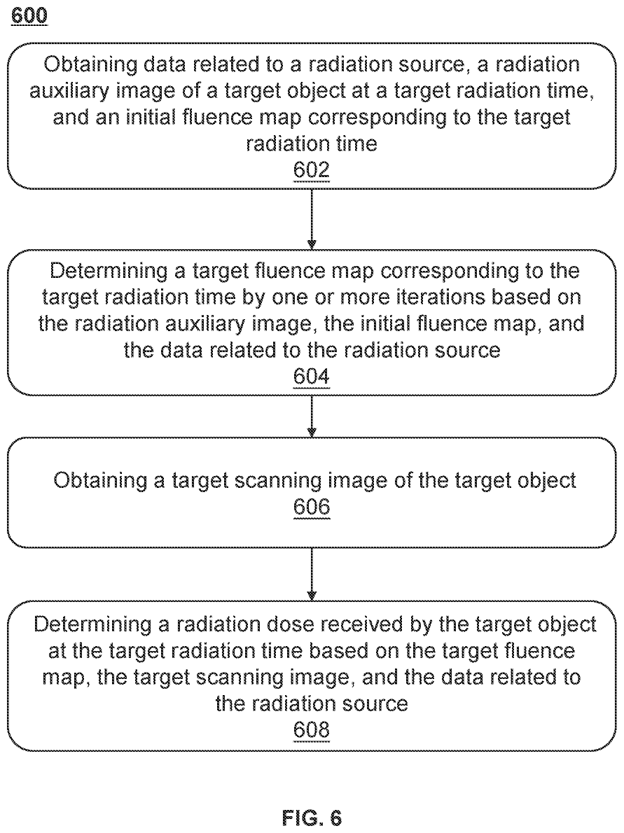

In some embodiments, the processing device 130 may further include a third determination module (not shown). The third determination module may obtain the radiation dose received by the target object during radiation at a plurality of radiation times and determine a total radiation dose received by the target object during the radiation based on the radiation dose received at the plurality of radiation times. More descriptions of the above modules may be found elsewhere in the present disclosure (e.g., FIGS. 6-9 and descriptions thereof).

It should be understood that the system and its modules shown in FIG. 5 may be implemented in various ways. For example, in some embodiments, the system and its modules may be implemented in hardware, software, or a combination thereof. The hardware part may be implemented by logic circuits; the software part may be stored in memory and executed by a suitable instruction execution system, such as a microprocessor or specially designed hardware. Those skilled in the art may understand that the above methods and systems can be implemented using computer executable instructions and/or contained in processor control code, such as providing such code on a carrier medium such as magnetic disk, CD or DVD-ROM, a programmable memory such as read only memory (Firmware), or a data carrier such as an optical or electronic signal carrier. The system and its modules of the present disclosure may be implemented not only by hardware circuits such as Very Large Scale Integration (VLSI) or gate arrays, semiconductors such as logic chips and transistors, or programmable hardware devices such as field programmable gate arrays and programmable logic devices but also by software executed by various types of processors, it may also be realized by the combination of the above hardware circuits and software (e.g., firmware).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the first obtaining module 510 and the second obtaining module 530 shown in FIG. 5 may be different modules in a system, or one module may realize the functions of the above two or more modules. As another example, each module may share one storage module, and each module can also have its own storage module. Such deformations are within the scope of protection of the present disclosure.

FIG. 6 is a flowchart illustrating an exemplary process for determining a radiation dose according to some embodiments of the present disclosure. In some embodiments, process 600 may be executed by the system 100. For example, the process 600 may be implemented as a set of instructions (e.g., an application) stored in a storage device (e.g., the storage device 150, and/or the memory 2820). In some embodiments, the processing device 130 (e.g., the processor 2810 of the computing device 2800, and/or one or more modules illustrated in FIG. 5) may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6 and described below is not intended to be limiting.

In 602, the data related to radiation source, the radiation auxiliary image of the target object at the target radiation time point, and the initial fluence map corresponding to the target radiation time point may be obtained. The operation may be executed by the first obtaining module 510.

In some embodiments, the data related to radiation source may be used to describe parameters of devices and/or assemblies related to ray delivery. The devices and/or assemblies may include a radioactive source, an accelerator, a collimator, or the like. Exemplary parameters may include a radiation beam energy, a radiation beam spot size, collimator physical parameters (e.g., a blade length, a blade thickness, a range of motion, or the like, of a multi-leaf collimator), etc. In some embodiments, the data related to radiation source may be obtained through calibration. For example, the radiation device may delivery radiation rays to a phantom (e.g., a water phantom), phantom data (e.g., a phantom thickness) and radiation auxiliary image data (e.g., EPID image data) may be acquired, and the data related to radiation source may be determined based on the acquired data.

It should be noted that before the target object (e.g., a cancer patient, a cancerous organ or tissue of the cancer patient, etc.) is treated by radiation rays, a radiation treatment plan may be determined. The treatment plan may indicate operations of the radiation device (e.g., the radiation device 110) throughout the radiation period. For example, the treatment plan may specify a plurality of nodes (also be referred to as control nodes), and each control node may correspond to a time point. The treatment plan may indicate a rotation angle of the gantry 113, moving positions of leaves of the multi-leaf collimator and/or the tungsten gate, a dose of rays emitted by the linear accelerator 111, or the like, at each time point. The radiation rays may be emitted at the control nodes (e.g., in a static intensity modulated radiation) or may be emitted continuously between two control nodes (e.g., in a dynamic intensity modulated radiation). Therefore, the target radiation time point may be a time point corresponding to the control node, or a time point between two control nodes. In some embodiments, at the target radiation time point, the radiation device (e.g., the radiation device 110) may begin or stop to emit radiation rays.

In addition, the radiation rays may be incompletely absorbed after passing through the target object, and a portion of the radiation rays may be received by an imaging assembly (e.g., the imaging assembly 115) after being attenuated. The imaging assembly 115 may be used to image the target object after the target object receives the radiation rays and an image may be generated. The obtained image may be used to assist the radiation. For example, the position of the target object may be confirmed (or verified), or the actual dose received by the target object may be determined. Therefore, after the radiation rays delivered by the radioactive source (e.g., the linear accelerator 111) pass through the target object, an image may be generated the imaging assembly (e.g., the imaging assembly 115), and the image may be referred to as the radiation auxiliary image.

In some embodiments, the radiation auxiliary image may include an EPID image. For example, the imaging assembly 115 may be an EPID. A detector in the EPID may detect the radiation rays passing through the target object and the detected radiation rays may be converted into electrical signals or digital signals (which may also be referred to as projection data). In some embodiments, the EPID image may be obtained by reconstruction based on the electrical signals or digital signals.

The fluence map may be referred to as an image that reflects a state of ray emission. For example, the fluence map may reflect the position of the collimator (e.g., the positions of the plurality of leaves of a multi-leaf collimator), the radiation beam intensity, or the like. In some embodiments, the initial fluence map corresponding to the target radiation time point may be a preset image. For example, the initial fluence map may be a preset CT image, or a CT image converted from another image with another modality (e.g., a PET image, an MRI image, etc.). As another example, the initial fluence map may be an image reconstructed based on data received by the imaging assembly after the radiation device emits radiation rays to the phantom. The initial fluence map may be pre-stored in the storage device (e.g., the storage device 150). The first obtaining module 510 may communicate with the storage device 150 to obtain the initial fluence map. In some embodiments, the initial fluence map corresponding to the target radiation time point may be obtained based on the radiation auxiliary image corresponding to the target radiation time point. For example, the first obtaining module 310 may obtain the initial fluence map by normalizing the radiation auxiliary image corresponding to the target radiation time point.

In 604, a target fluence map corresponding to the target radiation time point may be determined by one or more iterations based on the radiation auxiliary image, the initial fluence map, and the data related to radiation source. The operation 604 may be performed by the first determination module 520.

In some embodiments, the target fluence map may be an image that reflects state information of the radioactive source at the target radiation time point. It may be understood that the radiation rays may be captured by the detection assembly (e.g., the imaging assembly 115 of the radiation device 110) of the radiation device. The imaging assembly 115 may generate a corresponding image (e.g., the radiation device) based on the captured information. The radiation auxiliary image may reflect a ray dose (also be referred to as a radiation dose) received by the imaging assembly 115. According to the data related to radiation source that reflects the parameter of the devices and/or assemblies relating to the ray delivery and the initial fluence map that reflects the state information of the radioactive source, and/or other data (e.g., such as attenuation and/or absorption information of the radiation rays that pass through the target object), the first determining module 520 may determine the state information of the radioactive source at the target radiation time point. For example, the first determination module 520 may simulate a final target fluence map according to a physical motion process of the ray particles. The first determination module 520 may determine and update the fluence map repeatedly by the one or more iterations. Each iteration may be a simulation and updating process of the fluence map. After the iteration is terminated, the final fluence map may be determined as the target fluence map.

In some embodiments, the first determination module 520 may calibrate the radiation auxiliary image to obtain a calibrated radiation auxiliary image, and the target fluence map corresponding to the target radiation time point may be determined by the one or more iterations based on the calibrated radiation auxiliary image, the initial fluence map, and the data related to radiation source. In some embodiments, the calibration may include bad pixel calibration, dark current calibration, gain calibration, geometry calibration, etc., or any combination thereof.

In 606, a target scanning image of the target object may be obtained. The operation 606 may be performed by the second determination module 530.

In some embodiments, the target scanning image may correspond to the target radiation time point. The target scanning image may reflect the state of the target object at the target radiation time point. In some embodiments, during radiotherapy, the target object (e.g., a patient) may be moving autonomously (e.g., through physiological movements such as heartbeat, breathing, etc.). The state of the target object at the target radiation time point (e.g., a motion state of the chest of the patient due to breathing) may be used to guide the radiotherapy. For example, different motion states of the chest of the patient may affect dose distribution in the patient's body, and the position of the target region may change. Therefore, the target scanning image may be used in subsequent operations of the process 600 (e.g., be used to determine the radiation dose).

In some embodiments, the state of the target object at the target radiation time point may be considered as roughly the same as the state before the radiation time, therefore, the target scanning image of the target object corresponding to the target radiation time point may be considered to be similar to the scanning image of the target object before the radiation time (i.e., the scanning image of the target object before the radiation time may be used as the target scanning image of the target object corresponding to the target radiation time point). In some embodiments, the target scanning image may be an image obtained by scanning the target object before the target radiation time point. For example, before the radiotherapy, the target object may be scanned and imaged. The target scanning image may be determined from a plurality of obtained scanning images. In some embodiments, the target scanning image may be determined based on an X-ray imaging device (e.g., a computed radiograph (CR), a digital radiograph (DR), a computed tomography (CT), a mobile X-ray device (e.g., a mobile C-arm), a digital subtraction angiography scanner (DSA), an emission computed tomography (ECT), etc.). In some embodiments, the target scanning image may be determined based on a CT imaging device.

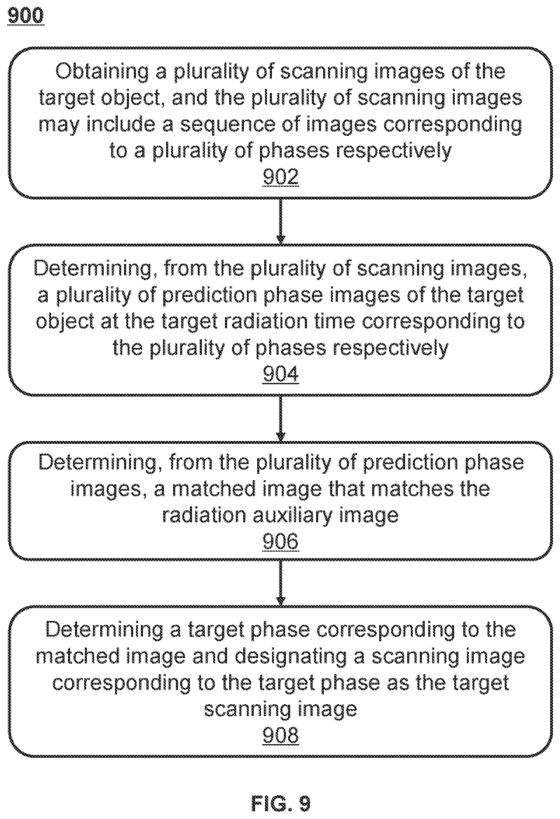

In some embodiments, the second obtaining module 530 may obtain a plurality of scanning images of the target object before the radiotherapy or at the target radiation time point. The plurality of scanning images may include a plurality of phase images of the target object corresponding to a plurality of phases. The second obtaining module 530 may determine a plurality of prediction phase images of the target object corresponding to the plurality of phases at the target radiation time point, and determine a matched image that matches the radiation auxiliary image from the plurality of prediction phase images. The matched image may be the prediction phase image most similar to the radiation auxiliary image corresponding to the target radiation time point. For example, the state of the target object in the matched image may be most similar to the state of the target object in the radiation auxiliary image. In some embodiments, a difference between the radiation auxiliary image and the matched image that matches the radiation auxiliary image may be less than a preset value. The second obtaining module 530 may designate a phase image corresponding to the phase of the matched image as the target scanning image. More descriptions of obtaining the target scanning image may be found elsewhere in the present disclosure (e.g., FIG. 9 and descriptions thereof).

In 608, the radiation dose received by the target object at the target radiation time point may be determined based on the target fluence map, the target scanning image, and the data related to radiation source. The operation 608 may be performed by the second determination module 540.

In some embodiments, the second determination module 540 may determine the radiation dose received by the target object at the target radiation time point using the Monte Carlo Method. In some embodiments, the Monte Carlo method may be used to simulate various physical processes of ray particles in the target object (e.g., scattering, attenuation, etc.). In some embodiments, the second determination module 540 may simulate a transport process of the ray particles using the Monte Carlo method. For example, under a parameter condition of devices and/or assemblies used for ray delivery as reflected by the data related to radiation source and a condition of the radioactive source reflected by the target fluence map, after passing through an inner region of the target object reflected by the target scanning image, a final dose distribution may be obtained. Based on the dose distribution, the second determination module 540 may determine the radiation dose received by the target object at the target radiation time point.

In some embodiments, the processing device 130 may further include a third determination module (not shown). The third determination module may obtain the radiation doses received by the target object during radiation at a plurality of target radiation time points and determine a total radiation dose received by the target object during the radiotherapy based on the radiation doses received at the plurality of target radiation time points. Each radiation time point may correspond to a gantry angle. The gantry angle may refer to a rotation angle of the gantry of the radiation device, which may be indicated by the control nodes specified in a radiation treatment plan. The radiation device may deliver ray(s) continuously to the target object at each gantry angle or deliver ray(s) within an angle range between the two gantry angles. The processing device 130 may determine the radiation dose received by the target object under each gantry angle and add these radiation doses to determine the total radiation dose received by the target object during the radiation.

In some embodiments, the processing device 130 may traverse the radiation doses received by the target object at the plurality of target radiation time points corresponding to the plurality of gantry angles in radiation, and determine the total radiation dose received by the target object during the radiation. It may be understood that, in some embodiments, the processing device 130 may calculate the plurality of radiation doses corresponding to the plurality of gantry angles in a fraction of the radiation, and sum the plurality of radiation doses to obtain the total radiation dose received by the target object during the radiation. In some embodiments, the processing device 130 may sequentially calculate the plurality of radiation doses corresponding to each gantry angle in the radiation. In some embodiments, during the process of sequential calculation, the processing device 130 may sum the calculated radiation doses, and finally obtain the total radiation dose. For example, after calculating two radiation doses (e.g., a first radiation dose and a second radiation dose) corresponding to two gantry angles (e.g., a first gantry angle and a second gantry angle), the two radiation doses may be summed, and after calculating a third radiation dose corresponding to a third gantry angle, a sum of the two previously obtained radiation doses may be summed with the newly calculated radiation dose (e.g., the third radiation dose), and so on, until the radiation doses corresponding to all gantry angles are calculated, and the total radiation dose may be obtained.

It should be noted that the above description of the process 600 is merely provided for the purposes of illustration, and not intended to limit the scope of the present disclosure. For those skilled in the art, various modifications and changes may be made to the process 600 under the guidance of the present disclosure. However, those variations and modifications do not depart from the scope of the present disclosure.

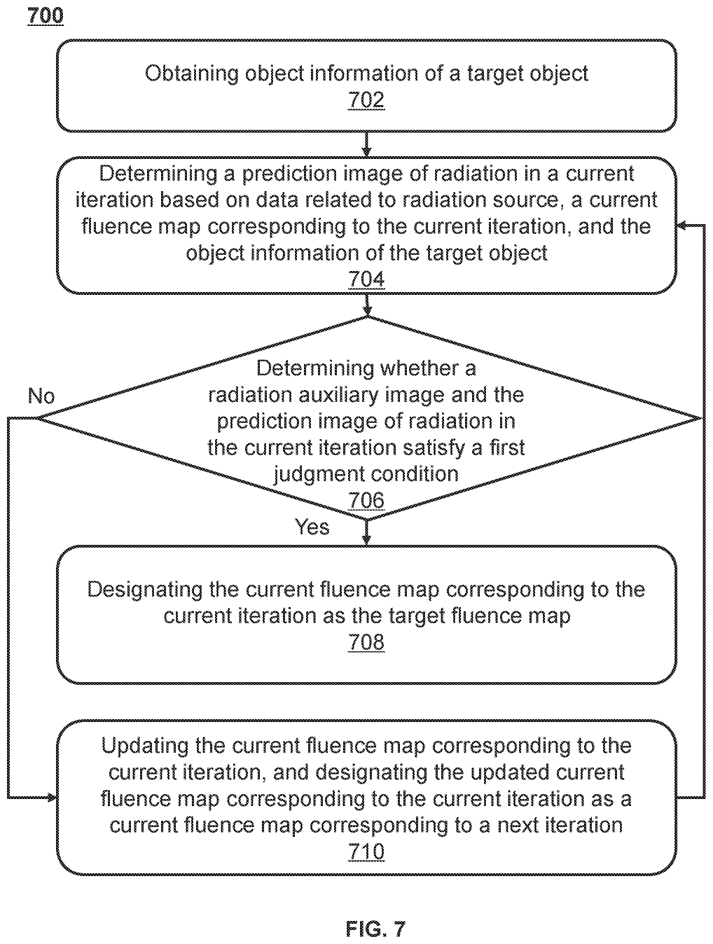

FIG. 7 is a flowchart illustrating an exemplary current iteration of the one or more iterations according to some embodiments of the present disclosure. In some embodiments, process 700 may be executed by the system 100. For example, the process 700 may be implemented as a set of instructions (e.g., an application) stored in a storage device (e.g., the storage device 150, and/or the memory 2820). In some embodiments, the processing device 130 (e.g., the processor 2810 of the computing device 2800, and/or one or more modules illustrated in FIG. 5) may execute the set of instructions and may accordingly be directed to perform the process 700. The operations of the illustrated process presented below are intended to be illustrative. In some embodiments, the process 700 may be accomplished with one or more additional operations not described and/or without one or more of the operations discussed. Additionally, the order of the operations of process 700 illustrated in FIG. 7 and described below is not intended to be limiting.

In 702, object information of the target object may be obtained.

In some embodiments, the object information of the target object may include scanning image information of the target object. The exemplary scanning image information may include CR image information, DR image information, CT image information, MRI image information, PET image information, or any combination thereof. In some embodiments, the object information may be obtained before the target radiation time point. For example, before the target radiation time point, a CT image obtained by performing CT scanning on the target object may be used as the object information of the target object. In some embodiments, the object information may be pre-stored in the storage device (e.g., the storage device 150). In some embodiments, the first determination module 320 may communicate with the storage device 150 to obtain the object information.

In 704, a prediction image of radiation in a current iteration may be determined based on the data related to radiation source, a current fluence map corresponding to the current iteration, and the object information of the target object.

In some embodiments, the prediction image of radiation may be used to indicate a prediction distribution map formed on the detection assembly (e.g., the imaging assembly 115 of the radiation device 110) after the movement of a plurality of particles of the radiation rays. In some embodiments, the first determination module 520 may determine a prediction image of radiation in the current iteration based on the data related to radiation source, the current fluence map corresponding to the current iteration, and the object information of the target object using the Monte Carlo Method.

For example, the first determination module 520 may simulate each particle in the radiation rays using the Monte Carlo method under the parameter condition of devices and/or assemblies used for ray delivery as reflected by the data related to radiation source and the condition of the radioactive source reflected by the target fluence map, through the transport process under a load free condition (e.g., there is no target object) and through the inner area of the target object reflected by the object information. The particles in each of the two cases may be captured by the detection assembly separately to obtain an image. An image intensity ratio of the two images (e.g., in the case with no target object and the case with the target object) may be referred to as a preset projection ratio. By multiplying the current fluence map corresponding to the current iteration by the preset projection ratio, the prediction image of radiation may be obtained.

It may be understood that the process of determining the target fluence map may be a process of a plurality of iterations. The fluence map corresponding to each iteration may be updated. For example, the current fluence map corresponding to the current iteration may be directly designated as the target fluence map in subsequent operations of the process 700 or may be updated for the next iteration. Therefore, a current fluence map corresponding to an iteration may be an updated fluence map corresponding to a previous iteration. A current fluence map corresponding to an initial iteration of the one or more iterations may be the initial fluence map.

In 706, whether the radiation auxiliary image and the prediction image of radiation in the current iteration satisfy a first judgment condition may be determined.

In some embodiments, the first judgment condition may include the prediction image of radiation in the current iteration being convergent to the radiation auxiliary image. The convergence may mean that a difference between the prediction image of radiation in the current iteration and the radiation auxiliary image is less than a preset threshold. The difference may include a difference between pixel values of corresponding pixels in two images. For example, the difference may be represented by a matrix. For example, a value in the matrix may represent a difference between the pixel values of corresponding two pixels. The difference between the two images being smaller than the preset threshold may mean that a modulus of the matrix representing the difference or an eigenvalue of the matrix is smaller than the preset threshold. The preset threshold may be predetermined or may be adjusted.

In some embodiments, if the radiation auxiliary image and the prediction image of radiation in the current iteration satisfy the first judgment condition (e.g., the prediction image of radiation in the current iteration being convergent to the radiation auxiliary image), the process 700 may proceed to operation 708. If the radiation auxiliary image and the prediction image of radiation in the current iteration do not satisfy the first judgment condition, the process 700 may proceed to operation 710.

In 708, in response to the radiation auxiliary image and the prediction image of radiation in the current iteration satisfying the first judgment condition, the current fluence map corresponding to the current iteration may be designated as the target fluence map.

In some embodiments, if the radiation auxiliary image and the prediction image of radiation in the current iteration satisfy the first judgment condition, the first determination module 520 may designate the current fluence map corresponding to the current iteration as the target fluence map. It may mean that the current fluence map corresponding to the current iteration may reflect the relevant state information of the radioactive source at the target radiation time may be used to determine the subsequence radiation dose.

In 710, in response to the radiation auxiliary image and the prediction image of radiation in the current iteration do not satisfy the first judgment condition, updating the current fluence map corresponding to the current iteration, and designating the updated current fluence map corresponding to the current iteration as a current fluence map corresponding to a next iteration.

In some embodiments, when the radiation auxiliary image and the prediction image of radiation in the current iteration do not satisfy the first judgment condition (i.e., the prediction image of radiation in the current iteration being not convergent to the radiation auxiliary image), the current fluence map corresponding to the current iteration may be updated. In some embodiments, the first determination module 520 may update the current fluence map corresponding to the current iteration.

In some embodiments, the first determination module 520 may determine a first difference between the radiation auxiliary image and the prediction image of radiation in the current iteration. The difference may be a first difference matrix between a first matrix representing the radiation auxiliary image and a second matrix representing the prediction image of radiation in the current iteration. The first difference matrix may be a difference between the first matrix and the second matrix. For example, the first difference matrix may be obtained by subtracting the second matrix from the first matrix or subtracting the first matrix from the second matrix. In some embodiments, the first difference matrix may be a quotient between the first matrix and the second matrix. For example, the first difference matrix may be obtained by multiplying the first matrix by the inverse of the second matrix or multiplying the second matrix by the inverse of the first matrix. In some embodiments, the first determination module 520 may update the current fluence map corresponding to the current iteration based on the first difference. For example, the first determination module 520 may sum the current fluence map corresponding to the current iteration and the first difference matrix. For instance, the first determination module 520 may sum the first difference matrix and a matrix representing the current fluence map corresponding to the current iteration. An image represented by a matrix obtained after the summation may be used as an updated fluence map.

In some embodiments, the first determination module may designate the updated current fluence map corresponding to the current iteration as the current fluence map corresponding to the next iteration, and enter the next iteration. In this way, one or more operations of 704, 706, 708, and 710 may be performed repeatedly. For example, in the next iteration, the operation 704 may be performed again, that is, the updated fluence map may be used as the current fluence map in the operations 704, 706, 708, or 710 corresponding to the next iteration.

It should be noted that the above description of process 700 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

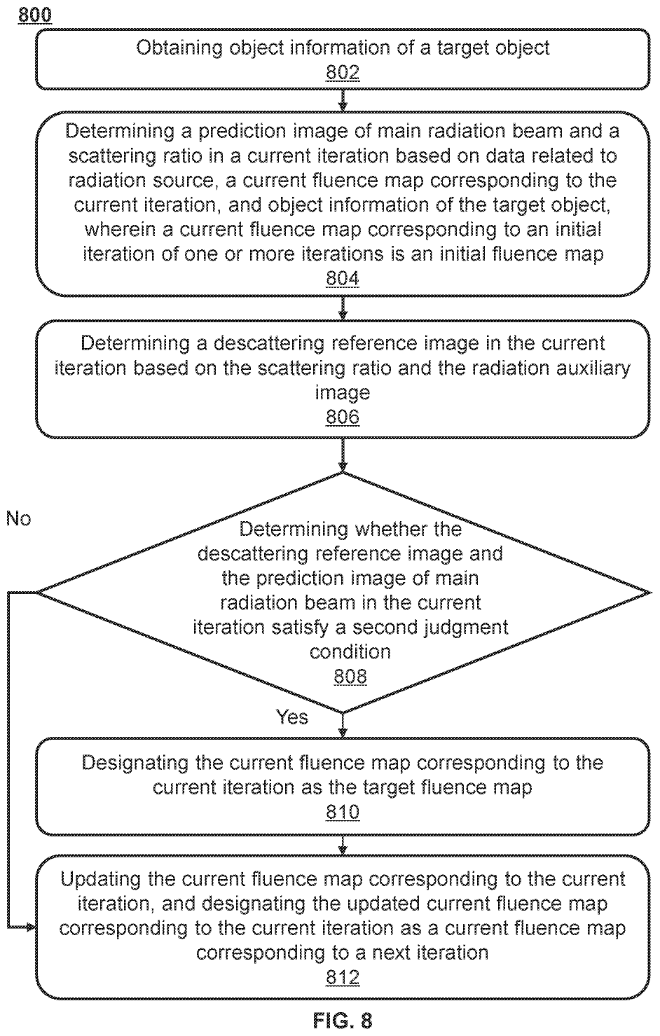

FIG. 8 is a flowchart illustrating another exemplary current iteration of the one or more iterations according to some embodiments of the present disclosure. In some embodiments, process 800 may be executed by the system 100. For example, the process 800 may be implemented as a set of instructions (e.g., an application) stored in a storage device (e.g., the storage device 150, and/or the memory 2820). In some embodiments, the processing device 130 (e.g., the processor 2810 of the computing device 2800, and/or one or more modules illustrated in FIG. 5) may execute the set of instructions and may accordingly be directed to perform the process 800. The operations of the illustrated process presented below are intended to be illustrative. In some embodiments, the process 800 may be accomplished with one or more additional operations not described and/or without one or more of the operations discussed. Additionally, the order of the operations of process 800 illustrated in FIG. 8 and described below is not intended to be limiting.

In 802, object information of the target object may be obtained.

In some embodiments, the operation 802 may be the same or similar to the operation 702 in process 700. More information about the operation 802 may refer to the operation 702, which may not be repeated here.

In 804, a prediction image of main radiation beam and a scattering ratio in the current iteration may be determined based on the data related to radiation source, a current fluence map corresponding to the current iteration, and object information of the target object. For example, the current fluence map corresponding to a first iteration may be the initial fluence map.

In some embodiments, the prediction image of main radiation beam may be an image formed by chief ray particles after removing scattering particles from the radiation rays. The scattering ratio may be a ratio between the amount of the scattering particles and the amount of the chief ray particles.

In some embodiments, the first determination module 520 may determine a prediction image of main radiation beam and a scattering ratio in the current iteration based on the data related to radiation source, the current fluence map corresponding to the current iteration, and the object information of the target object using the Monte Carlo Method. A current fluence map corresponding to an initial iteration of the one or more iterations is the initial fluence map. For example, the first determination module 520 may simulate each particle in the radiation rays using the Monte Carlo method, under the parameter condition of devices and/or assemblies used for ray delivery as reflected by the data related to radiation source and a condition of the radioactive source reflected by the current fluence map corresponding to the current iteration, and based on a plurality of physical motion processes of an inner region of the target object, and calculate a scattering ratio and an attenuation of particles passing through the target object, to obtain a prediction image of main radiation beam and a scattering image. The first determination module 520 may designate a ratio between the prediction image of main radiation beam and the scattering image as the scattering ratio. For example, the scattering ratio may be represented by SPRₙ(X,Y)=Sₙ(X,Y)/Pₙ(X,Y), in which n denotes an integer greater than 0, indicating the current iteration, Sₙ(X, Y) denotes the scattering image in the nth iteration, and Pₙ(X, Y) denotes the prediction image of main radiation beam in the nth iteration.

Similarly, the process of determining the target fluence map may include a plurality of iterations. The fluence map corresponding to each iteration may be updated. For example, the current fluence map corresponding to the current iteration may be designated as the target fluence map directly in the sequence operation of the process 700 or may be updated and used in the next iteration. Therefore, a current fluence map corresponding to a current iteration may be an updated fluence map in the previous iteration. The current fluence map corresponding to the first iteration may be the initial fluence map.

In 806, a descattering reference image in the current iteration may be determined based on the scattering ratio and the radiation auxiliary image.

In some embodiments, the descattering reference image may be an image determined by the chief ray particles captured by the detection assembly of the radiation device (e.g., the imaging assembly 115 of the radiation device 110). In some embodiments, the first determination module 520 may determine the descattering reference image Pₙ^{mea}(X, Y) according to the following Equation:

Pₙ^{mea}(X, Y)=(M(X, Y)/[1+SPRₙ(X, Y)])

wherein n denotes an integer greater than 0, indicating the current iteration, M(X, Y) denotes the radiation auxiliary image, and SPRₙ(X, Y) denotes the scattering image in the current nth iteration.

In 808, whether the descattering reference image and the prediction image of main radiation beam in the current iteration satisfy a second judgment condition may be determined.

In some embodiments, the second judgment condition may include the prediction image of main radiation beam in the current iteration being convergent to the descattering reference image in the current iteration. The convergence may mean that a difference between the prediction image of main radiation beam in the current iteration and the descattering reference image in the current iteration is less than a preset threshold. The difference may be related to a difference between pixel values of corresponding pixels in the two images. For example, the difference may be represented by a matrix. For example, a value in the matrix may represent a difference between the pixel values of corresponding two pixels of the two images. The difference being smaller than the preset threshold may mean that the modulus of the matrix representing the difference or an eigenvalue of the matrix is smaller than the preset threshold. The preset threshold value may be predetermined or may be adjusted.

In some embodiments, if the descattering reference image and the prediction image of main radiation beam in the current iteration satisfy a second judgment condition, the process 800 may proceed to operation 810. If the descattering reference image and the prediction image of main radiation beam in the current iteration do not satisfy the second judgment condition, the process 800 may proceed to operation 812.

In 810, in response to the descattering reference image and the prediction image of main radiation beam in the current iteration satisfying the second judgment condition, the current fluence map corresponding to the current iteration may be designated as the target fluence map.

In some embodiments, if the descattering reference image and the prediction image of main radiation beam in the current iteration satisfy the second judgment condition, the first determination module may designate the current fluence map corresponding to the current iteration as the target fluence map. It may mean that the current fluence map corresponding to the current iteration can reflect the state information of the radioactive source at the target radiation time point and may be used to determine the subsequence radiation dose.

In 812, in response to the descattering reference image and the prediction image of main radiation beam in the current iteration not satisfying the second judgment condition, the current fluence map corresponding to the current iteration may be updated, and the updated current fluence map corresponding to the current iteration may be designated as a current fluence map corresponding to a next iteration.

In some embodiments, if the descattering reference image and the prediction image of main radiation beam in the current iteration do not satisfy the second judgment condition, the current fluence map corresponding to the current iteration may be updated. In some embodiments, the first determination module 520 may update the current fluence map corresponding to the current iteration, and designate the updated current fluence map corresponding to the current iteration as a current fluence map corresponding to a next iteration.

In some embodiments, the first determination module 520 may determine a second difference between the descattering reference image and the prediction image of main radiation beam in the current iteration. The second difference may be a second difference matrix between a third matrix representing the descattering reference image in the current iteration and a fourth matrix representing the prediction image of main radiation beam in the current iteration. The second difference matrix may be a difference between the third matrix and the fourth matrix. For example, the second difference matrix may be obtained by subtracting the fourth matrix from the third matrix. In some embodiments, the second difference matrix may be a quotient between the third matrix and the fourth matrix. For example, the second difference matrix may be obtained by multiplying the third matrix by the inverse of the fourth matrix. In some embodiments, the first determination module 520 may update the current fluence map corresponding to the current iteration based on the second difference. For example, the first determination module 520 may sum the current fluence map corresponding to the current iteration and the second difference matrix. For instance, the first determination module 520 may sum the second difference matrix and the matrix representing the current fluence map corresponding to the current iteration, and designate an image represented by a matrix obtained after the summation as the updated fluence map.

It should be noted that the above description of process 800 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Based on the description of the foregoing flowchart, the radiation dose at the target radiation time point may be determined by using the information of the target scanning image corresponding to the target radiation time point. The target scanning image may reflect the state of the target object at the target radiation time point. At this time, the more accurate the state of the target object reflected by the target scanning image, the more accurate the result of the determined radiation dose.

FIG. 9 is a flowchart illustrating an exemplary process for obtaining a target scanning image of a target object according to some embodiments of the present disclosure. In some embodiments, process 900 may be executed by the system 100. For example, the process 900 may be implemented as a set of instructions (e.g., an application) stored in a storage device (e.g., the storage device 150, and/or the memory 2820). In some embodiments, the processing device 130 (e.g., the processor 2810 of the computing device 2800, and/or one or more modules illustrated in FIG. 5) may execute the set of instructions and may accordingly be directed to perform the process 900. The operations of the illustrated process presented below are intended to be illustrative. In some embodiments, the process 900 may be accomplished with one or more additional operations not described and/or without one or more of the operations discussed. Additionally, the order of the operations of process 900 illustrated in FIG. 9 and described below is not intended to be limiting.

In 902, a plurality of scanning images of the target object may be obtained, and the plurality of scanning images may include a sequence of images corresponding to a plurality of phases respectively.

In some embodiments, the plurality of scanning images may include a plurality of scanning images reflecting different motion states of the target object in one or more autonomous motion cycles. For example, the scanning images may include scanning images of the chest of a lung cancer patient in various states such as exhalation and inhalation in one or more breathing cycles. In the present disclosure, the state of the target object may also be referred to as a phase. The plurality of scanning images may correspond to different phases respectively. The breathing cycles may be divided into the plurality of phases. The scanning images may be 4D images, which may include a sequence of images corresponding to different time points, representing the scanning images corresponding to different phases in breathing cycles.

In some embodiments, the sequence of images corresponding to the plurality of phases may be captured within a preset time before a current fraction of the radiation to which the target radiation time point belongs.

In some embodiments, the plurality of scanning images may be determined by pre-scanning before the target radiation time point. For example, the target object may be scanned and imaged before the target radiation time point, to obtain the plurality of scanning images. As another example, the target object may be scanned and imaged in a preset time period (e.g., one week) before the target radiation time point, to obtain the plurality of scanning images. As a further example, the target object may be scanned and imaged periodically (e.g., once a week), to obtain the plurality of scanning images. In some embodiments, the plurality of scanning images may be reconstructed based on scanning data acquired in a most recent cycle before the fraction of the radiation to which the target radiation time point belongs. For example, the plurality of scanning images may be obtained before the beginning of a current radiation fraction. As another example, the plurality of scanning images may be obtained online. The online obtaining may refer to starting the radiation fraction after scanning the target object to obtain the images, and the target object does not need to leave the bed. It may be understood that the closer the scanning time is to the radiation time, the higher the accuracy of the obtained scanning image may be. Therefore, the plurality of online obtained scanning images may be more accurate. Further, the plurality of scanning images of the target object may be obtained online, and the images obtained at the beginning, during, or after the radiation may be highly matched with the state of the patient during the radiation, and thus, the radiation dose reconstructed based on the scanning images may be more accurate.

In some embodiments, the plurality of scanning images may include a plurality of four-dimensional computed tomography (4D-CT) images obtained by a 4D-CT imaging device. Since the 4D-CT provides a time dimension, the state of the target object at each time point may be reflected better. Compared with a time volume image obtained by a traditional CT, the 4D-CT image may reflect an actual state of the target object, and thus, the influence due to the motion of the target object may be eliminated in a greater extent. At the same time, since the acquisition of the plurality of scanning images may be performed before the radiation, the influence of posture changes (e.g., a patient's body shape change) and positioning of the target object may be eliminated.

In some embodiments, a target scanning image may be determined from the plurality of scanning images based on the radiation auxiliary image. In some embodiments, the target scanning image may be determined from the plurality of scanning images using one or more algorithms (e.g., an image processing algorithm) based on the radiation auxiliary image, which may not be limited in the present disclosure.

In 904, a plurality of prediction phase images of the target object at the target radiation time point corresponding to the plurality of phases may be determined from the plurality of scanning images.

In some embodiments, a prediction phase image may refer to a prediction image reflecting the state of the target object at the target radiation time point.

In some embodiments, in order to determine the plurality of prediction phase images corresponding to the plurality of phases, the second obtaining module 530 may obtain treatment planning information. The treatment planning information may be determined before the radiotherapy. For example, the treatment planning information of the target object may be determined based on a planning CT image. The treatment planning information may include state information of one or more treatment assemblies at the target radiation time point. For example, the treatment planning information may specify a plurality of control nodes, and each control node may correspond to a time point. The treatment planning information may include the state of each assembly of the radiation device at each control node, such as a rotation angle, a gantry speed, a moving position and a moving speed of the leaves and/or a tungsten gate of the collimator (e.g., a multi-leaf collimator), an intensity/energy of the rays emitted by the accelerator, a position of the treatment bed, etc. The second obtaining module 530 may determine planning delivery information at the target radiation time point based on the treatment planning information. The time corresponding to a control node may be the radiation time. At this time, the radiation device may start the ray delivery. Therefore, the second obtaining module 530 may determine planning delivery information at the target radiation time point based on the treatment planning information. The planning delivery information may include radiation beam angles and a segment parameter corresponding to each of the radiation beam angles. In some embodiments, the planning delivery information may further include a radiation beam intensity, a radiation beam conformal shape, a radiation dose, etc.

In some embodiments, for each phase of the plurality of phases, the second obtaining module 530 may obtain information relating to the phase. The information relating to the phase may include state information or stage information of the target object under the phase. For example, the information relating to the phase may include a stage of physiological movement (e.g., systolic phase of cardiac movement, diastolic phase of cardiac movement, etc.) of the target object (e.g., a patient, organ, or tissue of a patient), a posture of the target object (such as lying down, lying on the side, etc.), and/or a body shape of the target object, etc. In some embodiments, the second obtaining module may determine the prediction phase image corresponding to the phase based on the planning delivery information and the scanning image corresponding to the phase. For example, the second obtaining module 530 may simulate an initial state of each particle in the radiation rays when the imaging device delivers the radiation rays and a physical movement process of the particle before and after the particle passes through the target object (e.g., scattering, attenuation, etc.) based on the planning delivery information to obtain a state of the each particle when it is finally captured by the detection assembly of the radiation device (e.g., an energy, a speed, a motion direction, etc.) and a distribution result of all particles. The second obtaining module 530 may obtain the prediction phase image based on the above data (e.g., the state of the each particle, the distribution result of all particles, etc.).

In 906, a matched image that matches the radiation auxiliary image may be determined from the plurality of prediction phase images.

In some embodiments, the matched image may refer to a prediction phase image most similar to the radiation auxiliary image corresponding to the target radiation time point. For example, the state of the target object displayed in the matched image may be the most similar to the state of the target object displayed in the radiation auxiliary image. In some embodiments, the second obtaining module 530 may determine the matched image that matches the radiation auxiliary image using a feature matching algorithm. For example, the second obtaining module 530 may compare a feature distribution (e.g., a grayscale distribution feature) of the plurality of prediction phase images with a grayscale distribution feature of the radiation auxiliary image and choose the prediction phase image whose feature distribution is most similar to the grayscale distribution feature of the radiation auxiliary image as the matched image.

In some embodiments, the second obtaining module may determine first position information including a target tissue in the radiation auxiliary image, and second position information including the target tissue in each prediction phase image of the plurality of prediction phase images. The target tissue may refer to an identifiable tissue in the target object. For example, if the target object is the chest of lung cancer patient, the target object may be a tumor area or a lung organ. The first position information may be used to represent position of the target tissue in the radiation auxiliary image, which may be represented by a corresponding coordinate range. For example, the first position information may be represented by the coordinate range of pixels that belong to the target tissue in the radiation auxiliary image in the image coordinate system. Similar to the first position information, the second position information may be used to represent position of the target tissue in the prediction phase image, which may be represented by a corresponding coordinate range. For example, the second position information may be represented by the coordinate range of the pixels that belong to the target tissue in the prediction phase image in the image coordinate system.

In some embodiments, the second obtaining module 530 may determine the matched image that matches the radiation auxiliary image based on the first position information and the second position information. For example, the second obtaining module 530 may compare the first position information with the second position information corresponding to each prediction phase image. If the first position information matches the second position information corresponding to the prediction phase image (e.g., a difference between the coordinate ranges of the first position information and the second position information may be smaller than a preset range), the second obtaining module 530 may designate the prediction phase image as the matched image.

In some embodiments, the second obtaining module 530 may determine a third difference of each prediction phase image in the plurality of prediction phase images and the radiation auxiliary image. The third difference may refer to a difference between a matrix representing the radiation auxiliary image and a matrix representing the prediction phase image. For example, the third difference may be a matrix subtraction result obtained by subtracting the matrix representing the prediction phase image from the matrix representing the radiation auxiliary image. As another example, the third difference may be a matrix multiplication result obtained by multiplying the matrix representing the radiation auxiliary image by an inverse matrix of the matrix representing the prediction phase image. The second obtaining module 530 may determine a minimum value in the plurality of the third differences, and designate the prediction phase image corresponding to the minimum value as the matched image. For example, the second obtaining module 530 may determine a modulus or eigenvalue of the matrix subtraction result or matrix multiplication result representing a third difference, and designate the prediction phase image corresponding to the minimum modulus or eigenvalue as the matched image.

In 908, a target phase corresponding to the matched image may be determined and a scanning image corresponding to the target phase may be designated as the target scanning image.

In some embodiments, since the matched image is one of the plurality of the prediction phase images corresponding to the plurality of phases, after determining the matched image, the second obtaining module 530 may directly designate the phase corresponding to the matched image as the target phase. Then, the second obtaining module 530 may determine the scanning image corresponding to the target phase as the target scanning image.

It should be noted that the above description of process 900 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

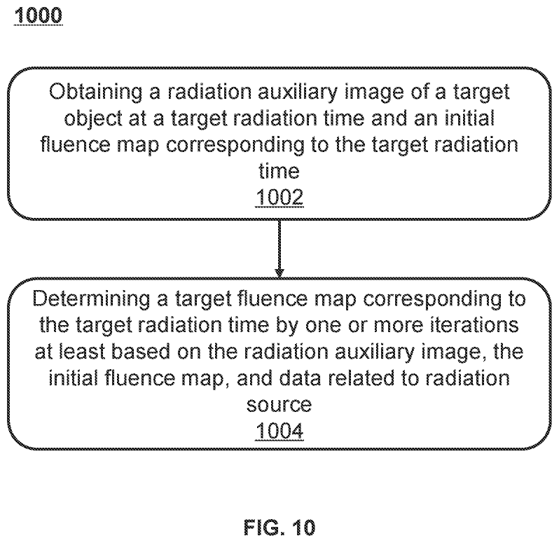

FIG. 10 is a flowchart illustrating an exemplary process for determining a fluence map according to some embodiments of the present disclosure. In some embodiments, process 1000 may be executed by the system 100. For example, the process 1000 may be implemented as a set of instructions (e.g., an application) stored in a storage device (e.g., the storage device 150, and/or the memory 2820). In some embodiments, the processing device 130 (e.g., the processor 2810 of the computing device 2800, and/or one or more modules illustrated in FIG. 5) may execute the set of instructions and may accordingly be directed to perform the process 1000. The operations of the illustrated process presented below are intended to be illustrative. In some embodiments, the process 1000 may be accomplished with one or more additional operations not described and/or without one or more of the operations discussed. Additionally, the order of the operations of process 1000 illustrated in FIG. 10 and described below is not intended to be limiting.

In 1002, a radiation auxiliary image of a target object at a target radiation time point and an initial fluence map corresponding to the target radiation time point may be obtained.

In 1004, a target fluence map corresponding to the target radiation time point may be determined by one or more iterations at least based on the radiation auxiliary image, the initial fluence map, and data related to radiation source. In some embodiments, the target fluence map may be used to reconstruct the radiation dose (as shown in FIG. 6), and/or may be used for other purposes, such as feature extraction, model training, etc., which may not be limited in the present disclosure.

More descriptions about the process 1000 may be found in FIG. 6 and the related descriptions, such as the operation 602 and the operation 604.

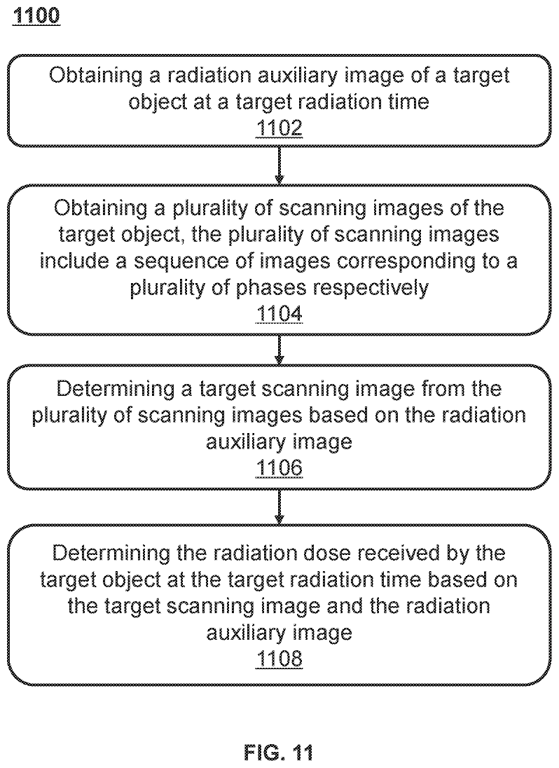

FIG. 11 is a flowchart illustrating an exemplary process for determining a radiation dose according to some embodiments of the present disclosure. In some embodiments, process 1100 may be executed by the system 100. For example, the process 1100 may be implemented as a set of instructions (e.g., an application) stored in a storage device (e.g., the storage device 150, and/or the memory 2820). In some embodiments, the processing device 130 (e.g., the processor 2810 of the computing device 2800, and/or one or more modules illustrated in FIG. 5) may execute the set of instructions and may accordingly be directed to perform the process 1100. The operations of the illustrated process presented below are intended to be illustrative. In some embodiments, the process 1100 may be accomplished with one or more additional operations not described and/or without one or more of the operations discussed. Additionally, the order of the operations of process 1100 illustrated in FIG. 11 and described below is not intended to be limiting.

In 1102, a radiation auxiliary image of a target object at a target radiation time point may be obtained.

More descriptions about the operation 1102 may be found in the operation 602 of FIG. 6 and the related descriptions in the present disclosure.

In 1104, a plurality of scanning images of the target object may be obtained, the plurality of scanning images may include a sequence of images corresponding to a plurality of phases respectively.

In some embodiments, the plurality of scanning images may include 4D-CT images obtained using an imaging device or online 4D-CT images.

More descriptions about the operation 1104 may be found in the operation 902 of FIG. 9 and the related descriptions in the present disclosure.

In 1106, a target scanning image may be determined from the plurality of scanning images based on the radiation auxiliary image.

In some embodiments, the processing device may determine a plurality of prediction phase images of the target object at the target radiation time point corresponding to the plurality of phases respectively from the plurality of scanning images, determine a matched image that matches the radiation auxiliary image from the plurality of prediction phase images, determine a target phase corresponding to the matched image, and/or designate a scanning image corresponding to the target phase as the target scanning image.

More descriptions about determining the target scanning image may be found in the operations 904-908 of FIG. 9 and the related descriptions in the present disclosure.

In 1108, the radiation dose received by the target object at the target radiation time point may be determined based on the target scanning image and the radiation auxiliary image.

In some embodiments, the processing device may obtain the target fluence map based on the radiation auxiliary image, and determine the radiation dose received by the target object at the target radiation time point based on the target fluence map and the target scanning image.

For example, the processing device may determine the target fluence map corresponding to the target radiation time point by the one or more iterations based on the radiation auxiliary image, the initial fluence map, and the data related to radiation source. More descriptions about determining the target fluence map may be found in the operation 604 of FIG. 6 and the related descriptions in the present disclosure.

In some embodiments, the processing device may determine the radiation dose received by the target object at the target radiation time point, based on the target fluence map, the target scanning image, and the data related to radiation source using the Monte Carlo Method. More descriptions about determining the radiation dose may be found in the operation 608 of FIG. 6 and the related descriptions in the present disclosure.

In some embodiments, the processing device may reconstruct the dose through various dose calculation engines to determine the radiation dose received by the target object at the target radiation time point based on the target fluence map and the target scanning image. In some embodiments, the processing device may reconstruct the dose using a convolution superposition algorithm or any other algorithm to determine the radiation dose received by the target object at the target radiation time point based on the target fluence map and the target scanning image.

According to some embodiments of the present disclosure, (1) by combining data related to radiation source with a forward iterative process, the computational complexity is reduced and the computational accuracy is improved; (2) by using 4D-CT image(s) in the dose reconstruction, the motion influence on dose reconstruction can be eliminated, and the influence of posture changes and positioning of the target object (e.g., the patient) can be eliminated. It should be noted that different embodiments may provide different beneficial effects. In different embodiments, the possible beneficial effects may be any one or a combination of the above, or any other possible beneficial effect.

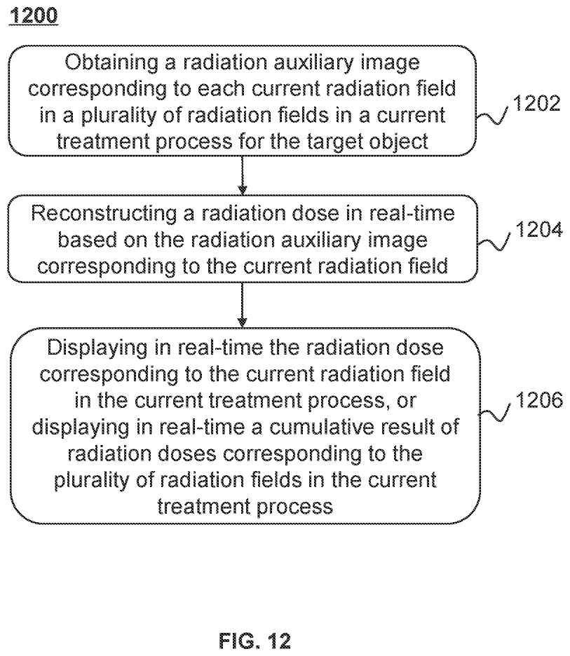

FIG. 12 is a flowchart illustrating an exemplary process for online radiation dose reconstruction according to some embodiments of the present disclosure. As shown in FIG. 12, process 1200 may include one or more of the following operations. In some embodiments, the process 1200 may be performed by a processing device or a system for dose verification.

In 1202, a radiation auxiliary image corresponding to each current radiation field in a plurality of radiation fields in a current treatment process for the target object may be obtained.

The target object may include a patient or any other medical object (e.g., an animal such as a mouse), etc. In some embodiments, the target object may be a part of the patient or other medical object, including an organ or tissue, such as a heart, a lung, a rib, an abdominal cavity, etc.

The target object may undergo one or more treatments according to a pre-specified treatment plan. The current treatment may be a treatment set by the treatment plan. For example, the current treatment may be a treatment process in which the target object can receive a certain dose of radiation at a period. As another example, the current treatment may be a treatment process in which the target object is treated for a while when the target object is located at a treatment bed. The present disclosure does not limit the delineation of the current treatment.

The radiation field refers to a region traversed by radiation. Radiation rays may travel in a straight line from a radiation source and may be scattered in a cone shape. Due to differences in an extent of the radiation treatment, a direction of the radiation rays, an angle of the radiation rays, a distance from the radiation rays to the target object (e.g., a tumor), and a projection of the tumor on the body surface, a plurality of radiation fields (or sub-fields) may be used to ultimately achieve radiation of an entire target area for treatment, avoiding endangering other organs while irradiating the target area more precisely. In some embodiments, treatment time corresponding to the plurality of radiation fields may be different, e.g., the plurality of radiation fields may be treated in a certain order based on the treatment plan, or the plurality of fields may be treated in a random order. The current radiation field refers to a radiation field for which radiation is currently delivered. In some embodiments, the current radiation field may be one or more radiation fields in the plurality of radiation fields.

The radiation auxiliary image may include a medical image obtained by an imaging assembly of a radiation device based on data generated by received ray(s) passing through the target object during the radiation treatment. For example, the radiation auxiliary image may be a medical image received by the imaging assembly (for example, the imaging assembly 115) and obtained based on data generated by received ray(s) after a radiation source (for example, the linear accelerator 111) passes through the target object at the radiation time point. In some embodiments, the radiation auxiliary image may include an electronic portal imaging device (EPID) image.

Each radiation field may correspond to a gantry angle. The gantry angle refers to a rotation angle of a gantry of the radiation device, which may be indicated by control nodes specified in a radiation treatment plan. The radiation treatment plan may specify a plurality of control nodes, each control node in the plurality of control nodes may correspond to a radiation field. The radiation treatment plan may include, at each control node, a state of various assemblies of a planned radiation device, the radiation device may deliver radiation rays to the target object at each gantry angle, or deliver the radiation rays continuously within an angle range including two gantry angles. In some embodiments, the obtained radiation auxiliary image may be determined based on the gantry angle. For example, for a normal type of intensity-modulated radiation treatment plan, a radiation auxiliary image may be output after accumulating pictures at each fixed gantry angle, and for an arc-based type of intensity-modulated radiation treatment plan, a radiation auxiliary image may be output after accumulating pictures in a range of the fixed gantry angle (e.g., 2°).

In some embodiments, the processing device may reconstruct the radiation auxiliary image based on data detected by detector(s) in an EPID that detect radiation rays passing through the target object and converting the detected radiation rays into electrical or digital signals (which may also be referred to as projection data). In some embodiments, in response to a completion of obtaining the radiation auxiliary image corresponding to the current radiation field, the radiation dose may be automatically reconstructed in real-time based on the radiation auxiliary image corresponding to the current radiation field. Automatically reconstructing the radiation dose in real-time may be clinically important, which may help guide the doctor to perform subsequent operation processes. In some embodiments, after the completion of obtaining the radiation auxiliary image corresponding to the current radiation field, in response to user operations (e.g., a click, a gesture, etc.), the processing device may reconstruct, based on the radiation auxiliary image corresponding to the current radiation field, the radiation dose in real-time.

In 1204, the radiation dose may be reconstructed in real-time based on the radiation auxiliary image corresponding to the current radiation field.

A real-time radiation dose reconstruction refers to a radiation dose reconstruction performed while the target object is treated on the treatment bed. In the process, the target object may not leave the treatment bed, or in other words, may not have to wait for too long, and the reconstruction of the radiation dose may be completed. Through the real-time radiation dose reconstruction, the doctor may get a quick overview of a treatment situation without letting the target object leave the treatment bed, which enables adjustment and optimization of the subsequent treatment plan. In some embodiments, the real-time radiation dose reconstruction may also be referred to as an online radiation dose reconstruction.

In some embodiments, the processing device may realize the radiation dose reconstruction based on the radiation auxiliary image corresponding to the current radiation field, data related to a radiation source, and a fluence map corresponding to the current radiation field. In some embodiments, a reconstructed radiation dose may be a two-dimensional dose or a three-dimensional dose obtained by accumulating a plurality of two-dimensional doses, which is not limited in the present disclosure. More description illustrating the reconstructed radiation dose may be found in the description of FIG. 5.

In some embodiments, after obtaining a real-time reconstructed radiation dose, the processing device may obtain a comparison result by comparing the real-time reconstructed radiation dose with an expected dose. The expected dose may be a predicted radiation dose that may be brought to the target object in the radiation treatment when determining the treatment plan. The comparison result may be a result obtained by comparing the radiation dose reconstructed in real-time with the radiation dose of the expected dose. For example, the comparison result may include the real-time reconstructed radiation dose being greater than the expected dose, the real-time reconstructed radiation dose being less than the expected dose, the real-time reconstructed radiation dose being close to the expected dose (e.g., being the same or a dose difference is less than a preset value), and the dose difference between the real-time reconstructed radiation dose and the expected dose.

In some embodiments, after determining the comparison result, the comparison result may be displayed by visualization, which facilitates the doctor to observe and guides the doctor to perform subsequent operations through a visualization interface, e.g., to view an underdose/overdose analysis result to optimize the treatment plan, etc. A specific visualization manner may include in a form of text, images, motion pictures, videos, etc., which is not limited in the present disclosure.

In some embodiments, the processing device may provide in real-time the underdose/overdose analysis result for a radiation target region or an organ at risk based on the comparison result. Underdose and overdose may be qualitative descriptions of the radiation dose corresponding to the current radiation field, which may be used to indicate whether a radiation dose of current radiation treatment is the expected dose. The underdose/overdose analysis result may be determined based on a comparison result. For example, a current real-time reconstructed radiation dose that is less than the expected dose may be considered to be underdose, or an amount of the current real-time reconstructed radiation dose that is less than the expected dose exceeding a threshold may be considered to be underdose. As another example, the current real-time reconstructed radiation dose that is greater than the expected dose may be considered to be overdose, or an amount of the current real-time reconstructed radiation dose that is greater than the expected dose exceeding a threshold may be considered to be an overdose.

The analysis result may be determined to be underdose or overdose only for the radiation target region or the organ at risk or for both the radiation target region and the organ at risk. For example, the real-time reconstructed radiation dose may be appropriate for the radiation target region but overdose for the organ at risk. In this case, whether the overdose exists may be determined based on the comparison result and the actual situation (e.g., a treatment status of the target object). In some embodiments, the analysis result may include an analysis result for the radiation target region and an analysis result for the organ at risk, which may be the same or different.

In some embodiments, the processing device may optimize, based on the comparison result, in real-time the treatment plan for a subsequent radiation field of the current radiation field in the current treatment process. For example, if the comparison result is that the real-time reconstructed radiation dose is greater than the expected dose, the radiation dose in a subsequent treatment plan may be reduced. By optimizing in real-time the treatment plan corresponding to the current treatment process, the radiation dose may be adjusted in time to avoid underdose or overdose in the treatment, which can minimize an additional radiation dose to other organs while performing the treatment on the target object better.

In 1206, the radiation dose corresponding to the current radiation field in the current treatment process may be displayed in real-time, or a cumulative result of radiation doses corresponding to the plurality of radiation fields in the current treatment process may be displayed in real-time.

A real-time display refers to displaying synchronously or after a short delay in a treatment process of the target object. For example, for the current treatment process, the radiation dose corresponding to the current radiation field may be displayed. The current radiation field may be a first radiation field in the current treatment process or a radiation field after completion of treatment in one or more radiation fields, and/or there may be unirradiated radiation fields after the current radiation field. The display of the radiation dose may be a display of a single radiation field. For example, the radiation dose corresponding to the current radiation field in the current treatment process may be displayed in real-time or the cumulative result of the radiation doses corresponding to the plurality of radiation fields that have been irradiated may be displayed in real-time.

In some embodiments, by obtaining in real-time the radiation auxiliary image corresponding to the current radiation field in the plurality of radiation fields in the current treatment process, the radiation dose may be reconstructed. In some embodiments, the radiation dose of the current treatment process may be fed back to the doctor in real-time, and the doctor may optimize the subsequent treatment plan based on the real-time displayed radiation dose to treat the patient better. At the same time, the real-time display of the radiation dose enables the doctor to be more proactive in performing the subsequent operations, such as making adjustments to the expected dose corresponding to the subsequent radiation field in the current treatment process, etc., to realize a real-time optimization of the treatment plan.

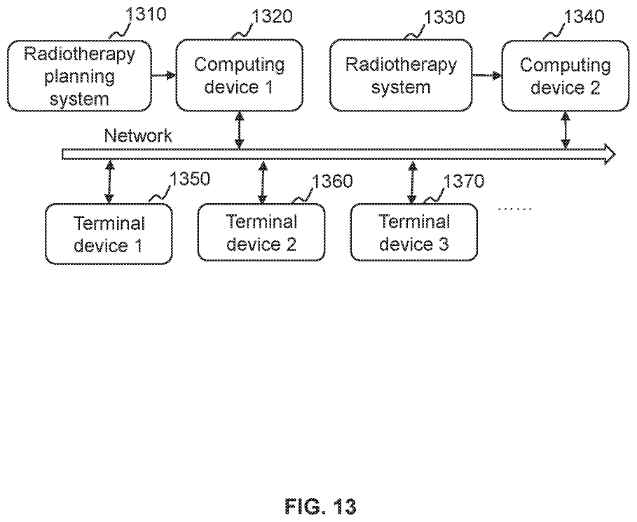

FIG. 13 is an exemplary schematic diagram illustrating a process of displaying in real-time a reconstruction result according to some embodiments of the present disclosure. As shown in FIG. 13, process 1300 may include one or more of the following operations. In some embodiments, the process 1300 may be performed by a processing device or a system for dose verification.

In some embodiments, a processing device may send a radiation auxiliary image to a radiotherapy planning system to reconstruct a radiation dose in the radiotherapy planning system in real-time. The reconstruction result of the radiation dose may be displayed in real-time via one or more terminal devices in communication with the radiotherapy planning system.

In an application scenario, a radiotherapy system may be in communication with a computing device 1, which may be configured to obtain the radiation auxiliary image. The computing device refers to a device with computing/data processing capabilities, such as a computer, a server, etc. The radiotherapy planning system may be on a computing device 2 in another room, and a radiation treatment plan may be formulated and/or adjusted on the computing device 2, and the formulated and/or adjusted radiation treatment plan (also referred to as the treatment plan) may be sent to a corresponding computing device or terminal device. For example, the formulated and/or adjusted radiation treatment plan may be sent to the computing device (e.g., the computing device 1) or the terminal device in communication with the radiotherapy system. For example, a user may initiate a request to view the radiation treatment plan via the computing device or the terminal device, and the computing device 2 may send the radiation treatment plan to a corresponding device based on a received request. The terminal device may include a smartphone, a tablet, a laptop, a desktop computer, etc.

In an application scenario, the radiotherapy planning system may be migrated (or set up) to the computing device 1 in communication with the radiotherapy system. In such a case, the radiation auxiliary image may be obtained directly from the computing device 1, instead of sending the radiation auxiliary image to the radiotherapy plan separately, which simplifies the workflow.

In some embodiments, the radiotherapy planning system, the computing device corresponding to the radiotherapy system, and the terminal device may be interconnected via a network. Exemplarily, as shown in FIG. 13, a radiotherapy planning system 1313 may be in communication with a computing device 1320 (which may be a wired connection or a wireless connection), and the radiation treatment system 1330 may be in communication with a computing device 1340 (which may also be a wired communication or a wireless communication), and the computing device 1320 and the computing device 1340 may be in communication with the network (e.g., a network 130). A terminal device 1350, a terminal device 1360, and a terminal device 1370 may also be in communication with the network to enable information exchange between the computing device 1320 and the computing device 1340 via the network. In some embodiments, the terminal device may be located in a different room from the computing device, and terminal device(s) and/or computing device(s) may be located in different rooms. For example, the computing device corresponding to the radiotherapy system may be located in room A, the computing device corresponding to the radiotherapy planning system may be located in room B, and the terminal device may be located in room C. Positions of the computing device and the terminal device, and communications between them may be set flexibly, which may not be limited in the present disclosure. In some embodiments, even if the doctor is not in front of the computing device corresponding to the radiotherapy system, the doctor may quickly learn about the situation of the radiation dose in radiation treatment, such as the computing device or the terminal device corresponding to the radiotherapy planning system, and then optimize the treatment plan in real-time based on a current radiation dose of the treatment plan.

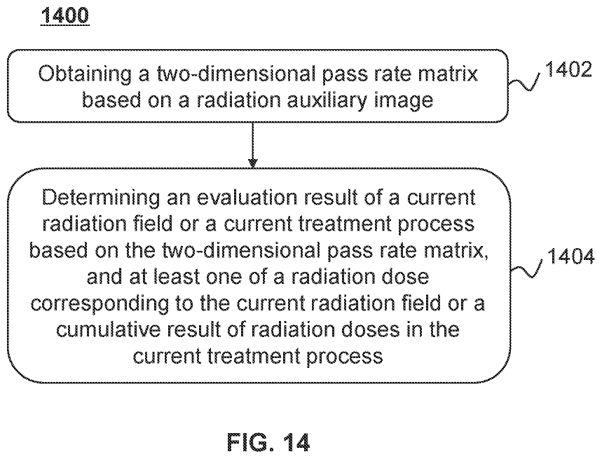

FIG. 14 is a flowchart illustrating an exemplary process for determining an evaluation result according to some embodiments of the present disclosure. As shown in FIG. 14, process 1400 may include one or more of the following operations. In some embodiments, process 1400 may be performed by a processing device or a system for dose verification.

In 1402, a two-dimensional pass rate matrix may be obtained based on a radiation auxiliary image.

The two-dimensional pass rate matrix refers to a matrix for evaluating an irradiation situation corresponding to a current radiation field. For example, after a treatment plan is determined, the treatment plan may include an expected examination, and the expected examination may have a corresponding expected dose. Each radiation field may have a corresponding expected dose. In an actual treatment process, after irradiation in the current radiation field, an actual radiation dose may be obtained by obtaining a corresponding radiation auxiliary image for real-time reconstruction, and a difference in radiation dose may be obtained by comparing the actual radiation dose with the expected radiation dose. The two-dimensional pass rate matrix is a metric for evaluating a similarity between the actual radiation dose and the expected radiation dose based on the difference in radiation dose.

In some embodiments, the processing device may obtain a two-dimensional dose image based on the radiation auxiliary image. For example, the radiation auxiliary image obtained at a corresponding current radiation field may be converted to a corresponding two-dimensional dose image using a model for dose reconstruction in a radiation field. The dose image may reflect an absolute dose distribution at a plane of an electronic portal imaging device (EPID) and is obtained by converting a grayscale pixel value to a dose value or simulating the grayscale pixel value. To convert the radiation auxiliary image to the two-dimensional dose image, either an empirical or a simulation model may be used. For example, a calibrated detector may include but is not limited to an ionization chamber, a miniature artifact-based, or a film-based detector used to convert EPID signals to dose. A detector response may be simulated or modeled through Monte Carlo technique or other empirical simulation techniques.

After the processing device obtains the two-dimensional dose image, the processing device may compare the radiation dose with an expected two-dimensional dose image of the radiation dose, determine the difference in radiation dose, and determine the two-dimensional pass rate matrix based on the the difference in radiation dose. For example, the processing device may directly designate the difference in radiation dose as the two-dimensional pass rate matrix, or determine the two-dimensional pass rate matrix based on the difference in radiation through a conversion.

In some embodiments, the two-dimensional pass rate matrix may be expressed as numerical values, e.g., 70, 89, 90, etc. The numerical values may be used to reflect the similarity between the actual radiation dose and the expected radiation dose, e.g., the larger the value, the higher the similarity. In some embodiments, a two-dimensional pass rate matrix threshold may be set. For example, the threshold may be 85. If the two-dimensional pass rate matrix exceeds the threshold, treatment corresponding to the current radiation field in a current treatment process may be considered to have met expectations.

In 1404, an evaluation result of the current radiation field or the current treatment process may be determined based on the two-dimensional pass rate matrix, and at least one of the radiation dose corresponding to the current radiation field or a cumulative result of radiation doses in the current treatment process.

The evaluation result may be used to reflect a treatment effect of the current radiation field or the current treatment process. The better the evaluation result, the better it reflects the treatment effect of the current radiation field or the current treatment (the closer it is to an expected treatment effect, the better the treatment effect may be considered). In some embodiments, the processing device may determine the evaluation result of the current radiation field based on the two-dimensional pass rate matrix and the radiation dose corresponding to the current radiation field. Furthermore, the processing device may determine the evaluation result of the current treatment process based on the two-dimensional pass rate matrix and the cumulative result of radiation doses in the current treatment process. In some embodiments, the processing device may add the evaluation result to a corresponding radiation dose based on the two-dimensional pass rate matrix, and the higher the two-dimensional pass rate matrix, the better the corresponding evaluation result.

In some embodiments, by determining the evaluation result of the current radiation field or the current treatment process, the doctor may know about the treatment effect of the current radiation field or the current treatment process. Therefore, the doctor may assess, based on the evaluation result of the current radiation field, whether treatment in other subsequent radiation field(s) is appropriate and make a corresponding adjustment to the treatment plan if needed, and/or may judge whether the treatment plan of further treatment is appropriate and make a corresponding adjustment to the treatment plan if needed to treat the patient better.

Quality assurance (QA) may also be referred to as patient specific quality assurance, which is an important part of radiation treatment. A commonly used manner for QA may include using a device for dose verification to measure a point dose, a surface dose, etc., when executing a plan and assessing an effect of executing the plan by comparing a measured dose with a theoretically calculated dose.

Exemplarily, the QA may be typically achieved by using an ionization chamber, a semiconductor probe to measure the point dose of a phantom, and using the device for dose verification to measure the surface dose. However, errors in operating an accelerator may be caused by positioning variation and body posture variation of a patient in treatment in each radiation fraction, which makes online verification fail to be implemented using the commonly used manner for QA in an actual treatment process.

With the development of medical technology, in vivo dose monitoring of the patient in radiation treatment has been more widely implemented, and an existing in vivo manner may mainly include the following two types. A first type of manner may include a measurement of a point dose on a body surface of the patient. For the first type, a thermoluminescent dosimeter (TLD), a semiconductor detector (a diode), etc., may be commonly used to measure the point dose of radiation irradiated on the patient and radiation emitted from the body surface of the patient. In some embodiments, the first type of manner may have too few measurement points, lack spatial distribution, and information reflected may be single. A second type of manner may include using an electronic portal imaging device (EPID) to measure a transmitted radiation image of the patient. The EPID may measure a sequence of the transmitted radiation image of the patient to perform dose verification in a treatment process. A prerequisite of the second type of manner may include calibrating the obtained images from the EPID. At the same time, accurate modeling of an EPID response may also be required.

In the treatment process, the EPID may measure the sequence of the transmitted radiation image of the patient and compare it with an expected image (or a baseline image), which enables in vivo dose verification. The expected image (or the baseline image) of the EPID may be obtained by combining dose calculation with image information (CT, MR, etc.) of the patient obtained at the beginning of the treatment process. Using a gamma pass rate algorithm for assessing a similarity between two sets of images, a quantitative assessment of the similarity may be performed on an EPID measured image and the expected image (or the baseline image), thus an assessment result may be obtained, then the effect of executing the plan may be determined based on the assessment result.

However, in an actual treatment process, errors in operating an accelerator may be caused by positioning variation and body posture variation of a patient, which often brings a certain deviation in a dose received by the patient from what is expected, and the deviation may be caused by different factors. Therefore, a type of dose error received by the patient may be different. Traditional manners may not determine the type of the dose error during treatment in a radiation fraction.

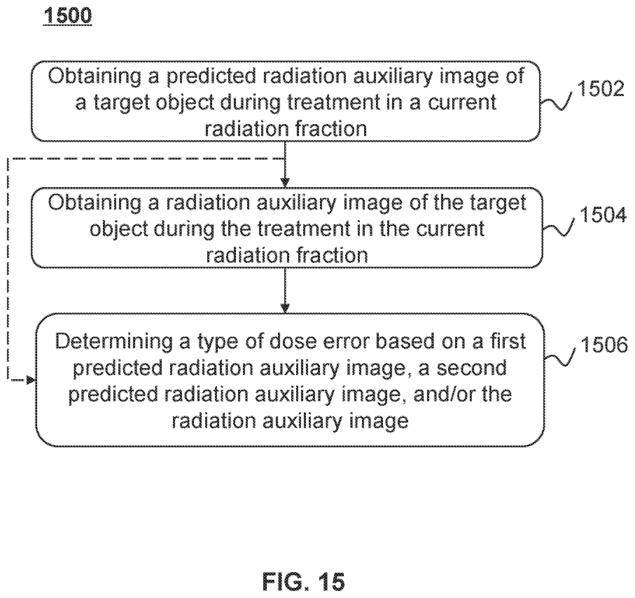

FIG. 15 is a flowchart illustrating an exemplary process for determining a type of dose error according to some embodiments of the present disclosure. As shown in FIG. 15, process 1500 may include one or more of the following operations. In some embodiments, the process 1500 may be performed by a processing device or a system for dose verification.

In 1502, a predicted radiation auxiliary image of a target object during treatment in a current radiation fraction may be obtained.

The predicted radiation auxiliary image may include a first predicted radiation auxiliary image and a second predicted radiation auxiliary image. The first predicted radiation auxiliary image may be obtained based on an initial medical scanning image of the target object, and the second predicted radiation auxiliary image may be obtained based on a medical scanning image of the target object before or during the treatment in the current radiation fraction. In some embodiments, the medical scanning image may include a CT image, an MR image, etc., which is acquired before or during the treatment is executed during the current radiation fraction.

Typical procedures of radiation treatment may usually include the following operations. Medical imaging scanning may be performed on the target object to obtain the initial medical scanning image (including but not limited to, a computed tomography (CT) image, a magnetic resonance (MR) image, etc.), a target region and an organ at risk may be outlined and an initial treatment plan may be made based on the initial medical scanning image, wherein the initial treatment plan may include treatment in a plurality of radiation fractions and parameters related to treatment in each radiation fraction in the plurality of radiation fractions, i.e., treatment plans of the treatment in the plurality of radiation fractions. The typical procedures may also include the quality assurance, implementation of the treatment in the radiation fraction, etc. The initial medical scanning image refers to the medical scanning image of the target object that is obtained to make the initial treatment plan before performing the radiation treatment process on the target object.

After obtaining the initial treatment plan, the first predicted radiation auxiliary image corresponding to the treatment in each radiation fraction may be obtained based on the treatment plan of the treatment in each radiation fraction in the initial treatment plan and the initial medical scanning image of the target object. That is, the first predicted radiation auxiliary image may be obtained based on the initial medical scanning image of the target object combined with the radiation dose in the treatment plan by calculation, which may be used to characterize a theoretical EPID image of radiation rays received by the target object during the treatment in each radiation fraction. Further, after obtaining the first predicted radiation auxiliary image corresponding to the treatment in each radiation fraction, the first predicted radiation auxiliary image corresponding to the treatment in each radiation fraction may be saved, so that the first predicted radiation auxiliary image may be directly obtained when performing treatment in a subsequent radiation frraction.

Alternatively, it should be noted that, if the parameters related to the treatment in each radiation fraction are the same, a first preset EPID image corresponding to the treatment in each radiation fraction may also be the same. That is, when determining the first predicted radiation auxiliary image corresponding to the treatment in each radiation fraction based on the initial treatment plan and the initial medical scanning image, the first predicted radiation auxiliary image corresponding to treatment in a current radiation fraction may be obtained based on the treatment plan in treatment in any radiation fraction and the initial medical scanning image, and the first predicted radiation auxiliary image may be designated as the first predicted radiation auxiliary image corresponding to the treatment in each radiation fraction.

During the radiation fraction and before radiation beams are delivered, the medical image scanning may be performed on the target object to obtain the medical scanning image (including but not limited to, a computed tomography (CT) image, a magnetic resonance (MR) image, etc.) during the treatment in the current radiation fraction, and the second predicted radiation auxiliary image of the target object corresponding to the treatment in the current radiation fraction may be calculated based on the medical scanning image during the treatment in the current radiation fraction combined with a radiation dose applied in the treatment in the current radiation fraction. The radiation dose applied in the treatment in the current radiation fraction may be obtained from the initial treatment plan. Certainly, in the case of the initial treatment plan being updated, the treatment plan of the treatment in the current radiation fraction may also be determined based on a latest treatment plan of the target object, and the radiation dose applied in the treatment in the current radiation fraction may also be further determined.

Optionally, when the target object does not receive medical image scanning during the treatment in the current radiation fraction, a medical scanning image of the target object of treatment in a previous radiation fraction may also be designated as the medical scanning image during the treatment in the current radiation fraction. That is, when the target object does not receive the medical image scanning during the treatment in the current radiation fraction, a recently scanned medical scanning image of the target object may also be designated as the medical scanning image during the treatment in the current radiation fraction. The recently scanned medical scanning image may include the medical scanning image of the treatment in the previous radiation fraction, a medical scanning image of treatment in a last previous radiation fraction, or a medical scanning image obtained during treatment not in a radiation fraction on the target object, etc., which may not be limited in the embodiments of the present disclosure, as long as the medical scanning image can characterize information related to recent positioning and body posture of the target object.

In 1504, a radiation auxiliary image of the target object during the treatment in the current radiation fraction may be obtained.

Exemplarily, when performing the treatment in the current radiation fraction on the target object, the EPID may be used to measure the transmitted radiation image of the patient, and the radiation auxiliary image of the target object during the treatment in the current radiation fraction may be obtained. The radiation auxiliary image may be used to characterize imaging of the target object on the EPID after the radiation dose that is actually received by the target object after transmitting through the target object during the treatment in the current radiation fraction.

In 1506, the type of the dose error during the treatment in the current radiation fraction may be determined based on the first predicted radiation auxiliary image, the second predicted radiation auxiliary image, and/or the radiation auxiliary image.

During the treatment in the radiation fraction, a period of time may exist between treatment in each radiation fraction, thus the body posture and the positioning of the target object may be different. When using a radiation device during a long term, errors may also exist in the accelerator that is used to apply the radiation rays, thus, under influence of various factors, a radiation dose received by the target object during the treatment in the radiation fraction may deviate from a planning radiation dose. When there is a dose error, to ensure accurate treatment of the target object, a clear determination of a type of the dose error may be required to adjust the treatment plan according to the determined type of the dose error or to make adjustments to parameters of the accelerator in the radiation device.

The first predicted radiation auxiliary image may be obtained based on the initial treatment plan of the target object that is determined based on the initial medical scanning image of the target object. Thus, during the whole treatment process, if the body posture of the target object and the positioning of the target object during the treatment in each radiation fraction do not change, the predicted radiation auxiliary image of the target object during the treatment in each radiation fraction may remain consistent. However, during the treatment in the radiation fraction, to a large extent, changes in the body posture of the target object may be subtle or even large, and it is difficult to keep the positioning of the target object exactly the same. Therefore, for the treatment in each radiation fraction, a difference between the second predicted radiation auxiliary image determined based on the medical scanning image of the target object during the treatment in the current radiation fraction and the first predicted radiation auxiliary image determined based on the initial treatment plan, which may be used to characterize the changes in the body posture and positioning of the target object.

Further, since the second predicted radiation auxiliary image of the target object is obtained based on the medical scanning image of the target object during the treatment in the current radiation fraction, the second predicted radiation auxiliary image may characterize the predicted radiation auxiliary image of the target object more accurately. The second predicted radiation auxiliary image may be used in conjunction with the radiation auxiliary image of the target object during the treatment in the current radiation fraction to further determine whether the error exists in the accelerator of the radiation device.

In such a case, the type of the dose error may include at least a first type and a second type. The first type of dose error may be configured to characterize one or more dose errors caused by positioning variation and/or body posture variation of the target object, and the second type of dose error may be configured to characterize one or more dose errors caused by variation of one or more performance parameters of a treatment accelerator during the treatment in the current radiation fraction.

Further, a computer device may determine, based on the first predicted radiation auxiliary image and the second predicted radiation auxiliary image of the target object, whether the type of the dose error during the treatment in the current radiation fraction is the first type of dose error, and determine, based on the second predicted radiation auxiliary image and the radiation auxiliary image, whether the type of the dose error during the treatment in the current radiation fraction is the second type of dose error.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, operation 1504 may be omitted, i.e., 1506 may be directly performed after 1502, and in determining the type of dose error based on a first predicted radiation auxiliary image and a second predicted radiation auxiliary image, the type of dose error may be caused by variation of the target object. As another example, in 1504-1506, the type of dose error may be determined based on the radiation auxiliary image. Specifically, the second predicted radiation auxiliary image may be determined based on the acquired radiation auxiliary image, and the medical scanning image(s) (e.g., a CT image, an MR image, etc.) of the current radiation fraction, and the type of dose error of the current radiation fraction may be determined, in which the type of dose error may be caused by the execution of the treatment accelerator. As a further example, through 1502-1506, the dose error caused by the variation of the target object and the dose error caused by the execution of the treatment accelerator may be determined simultaneously.

It should be noted that the first type of dose error may also be configured to characterize one or more dose errors caused by variation of one or more performance parameters of the treatment accelerator during the treatment in the current radiation fraction, and the second type of dose error may be configured to characterize one or more dose errors caused by the positioning variation and/or the body posture variation of the target object. In such a case, the computer device may determine, based on the second predicted radiation auxiliary image and the radiation auxiliary image of the target object, whether the type of the dose error during the treatment in the current radiation fraction is the first type of dose error, and determine, based on the first predicted radiation auxiliary image and the second predicted radiation auxiliary image of the target object, whether the type of dose error during the treatment in the current radiation fraction is the second type of dose error.

In the above-described manner for determining the type of the dose error, the computer device may determine the type of the dose error by obtaining the first predicted radiation auxiliary image and the second predicted radiation auxiliary image of the target object during the treatment in the current radiation fraction. The first predicted radiation auxiliary image may be obtained based on the initial medical scanning image of the target object. The second predicted radiation auxiliary image may be obtained based on the medical scanning image of the target object during the treatment in the current radiation fraction. Then, the computer device may obtain the radiation auxiliary image of the target object during the treatment in the current radiation fraction, and further determine the type of the dose error during the treatment in the current radiation fraction based on the first predicted radiation auxiliary image, the second predicted radiation auxiliary image, and the radiation auxiliary image. That is, in some embodiments of the present disclosure, the computer device may determine different types of the dose error of the target object based on the predicted radiation auxiliary image and the radiation auxiliary image obtained in different states. As the predicted radiation auxiliary image and the radiation auxiliary image in different states may characterize different states of the target object and different states of the radiation device, factors that cause the dose error may be accurately determined, i.e., a source of the dose error may be determined, to obtain the type of the dose error, which improves accuracy and rationality in determining the type of the dose error. In addition, since the manner in the present disclosure may locate the source of the dose error accurately, timely adjustments may be made to the dose error, thereby reducing the dose error and improving accuracy of the treatment in the radiation fraction.

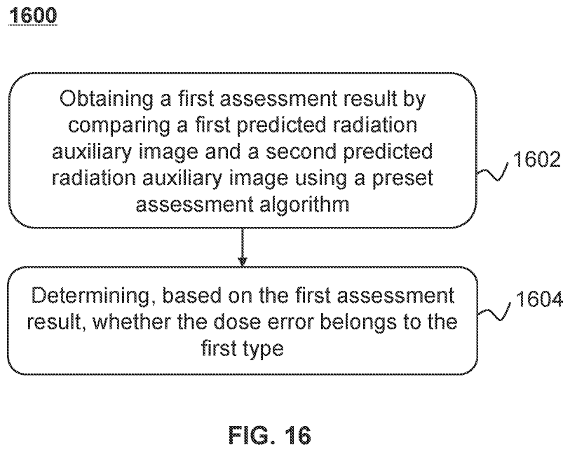

FIG. 16 is a flowchart illustrating an exemplary process for determining a first type of dose error according to some embodiments of the present disclosure. As shown in FIG. 16, process 1600 may include one or more of the following operations. In some embodiments, process 1600 may be performed by a processing device or a system for dose verification.

In 1602, a first assessment result may be obtained by comparing a first predicted radiation auxiliary image and a second predicted radiation auxiliary image using a preset assessment algorithm.

The first assessment result may be configured to characterize a similarity between the first predicted radiation auxiliary image and the second predicted radiation auxiliary image. The preset assessment algorithm may be configured to perform a quantitative assessment of the similarity between the first predicted radiation auxiliary image and the second predicted radiation auxiliary image to obtain an assessment result of the similarity. Exemplarily, a computer device may obtain the first assessment result by performing similarity evaluation using the preset assessment algorithm based on the first predicted radiation auxiliary image and the second predicted radiation auxiliary image.

In some embodiments, the preset assessment algorithm may be a gamma pass rate assessment algorithm, the gamma pass rate assessment algorithm may use a gamma function to perform a comparative calculation on two images to obtain a gamma pass rate and evaluate similarity between the two images based on the gamma pass rate. Accordingly, in some embodiments, the gamma pass rate assessment algorithm may be used to perform the comparative calculation on the first predicted radiation auxiliary image and the second predicted radiation auxiliary image to obtain a gamma pass rate used to characterize the similarity between the first predicted radiation auxiliary image and the second predicted radiation auxiliary image. Then the gamma pass rate may be designated as the first assessment result between the first predicted radiation auxiliary image and the second predicted radiation auxiliary image. That is, in some embodiments, the similarity between the two images may be characterized by the gamma pass rate, and the gamma pass rate may be designated as the first assessment result.

In the principle of the gamma pass rate assessment algorithm, for each corresponding pixel point in the first predicted radiation auxiliary image and the second predicted radiation auxiliary image, the corresponding pixel point may be input into the gamma function to obtain an image gamma value of the corresponding pixel point. Based on a gamma assessment criterion, if the image gamma value is less than or equal to 1, it may indicate that the corresponding pixel point passes the assessment, and if the image gamma value is greater than 1, it may indicate that the corresponding pixel point does not pass the assessment. A ratio of pixel points that pass the assessment to a total count of pixel points in an EPID image may be calculated, i.e., a quotient of a count of the pixel points that pass the assessment to the total count of pixel points in the EPID image may be calculated, the quotient may be designated as the gamma pass rate of the first predicted radiation auxiliary image and the second predicted radiation auxiliary image.

Alternatively, the gamma function may include a plurality of preset assessment parameters, different combinations of values of the plurality of assessment parameters may be used to characterize different assessment conditions. For example, the assessment parameter may include a dose tolerance and a distance tolerance between two pixel values, such as a dose tolerance of 2% and a distance tolerance of 2 millimeters. If a set value of each assessment parameter in the plurality of assessment parameters varied, an obtained gamma value of the image between the two pixel points may vary, which indicates that in the case where the assessment conditions are different, the similarity between the two images may be different.

Exemplarily, if the assessment condition is that a dose tolerance being 2% and a distance tolerance being 2 millimeters, the corresponding gamma pass rate may be considered to have a high similarity between the two images when it reaches 95% (including greater than or equal to 95%). It should be noted that the set value of each assessment parameter in the embodiments of the present disclosure is not specifically limited, and the user may flexibly adjust a value of each assessment parameter in the actual application.

In some embodiments, after obtaining the gamma pass rate, the first assessment result may be further obtained based on the gamma pass rate. Exemplarily, the gamma pass rate may be compared with a preset gamma pass rate threshold to obtain the first assessment result that is used to characterize whether the first predicted radiation auxiliary image and the second predicted radiation auxiliary image are similar. The first assessment result may be a particular number, character, string, or a combination thereof. For example, if the gamma pass rate is greater than or equal to the preset gamma pass rate threshold (such as 95% as described above), the first assessment result, such as character 1, may be obtained to characterize that the first predicted radiation auxiliary image and the second predicted radiation auxiliary image are similar. If the gamma pass rate is less than the preset gamma pass rate threshold (such as 95% as described above), the first assessment result, such as character 0, may be obtained to characterize that the first predicted radiation auxiliary image and the second predicted radiation auxiliary image are unsimilar.

That is, in some embodiments, if the similarity between the two images reaches a certain similarity threshold, the two images may be considered to be similar to each other, and accordingly, the first assessment result for characterizing the similarity between the two images may be obtained.

Furthermore, it should be noted that the gamma pass rate assessment algorithm is only exemplified as one of the preset assessment algorithms, which may include other algorithms for determining the similarity between the two images, such as a relative dose bias algorithm, a distance to agreement (DTA) algorithm, etc. Certainly, assessment metrics such as image likeness may also be used to determine the similarity between the two images.

In 1604, whether the dose error during treatment in a current radiation fraction belongs to a first type of dose error may be determined based on the first assessment result.

The first type of the dose error may be used to characterize one or more dose errors caused by positioning variation and body posture variation of the target object.

Exemplarily, if the first assessment result is the similarity (such as the gamma pass rate as described above) of the first predicted radiation auxiliary image and the second predicted radiation auxiliary image, the similarity may be compared to a first similarity threshold. If the similarity is less than or equal to the first similarity threshold, it may indicate that the similarity between the first predicted radiation auxiliary image and the second predicted radiation auxiliary image is low, which in turn may indicate that the first type of the dose error caused by the positioning variation and the body posture variation of the target object may exist during the treatment in the current radiation fraction, i.e., the dose error during the treatment in the current radiation fraction may belong to the first type of dose error.

On the contrary, if the first assessment result is to characterize that the similarity between the first predicted radiation auxiliary image and the second predicted radiation auxiliary image is greater than the first similarity threshold, it may indicate that the first type of the dose error caused by the positioning variation and the body posture variation of the target object does not exist during the treatment in the current radiation fraction, i.e., it may indicate that the dose error during the treatment in the current radiation fraction does not belong to the first type of dose error.

Exemplarily, if the first assessment result is a result of numbers, characters, or combinations thereof characterizing whether the first predicted radiation auxiliary image and the second predicted radiation auxiliary image are similar or match, whether the first assessment result is a preset assessment result may be determined. If the first assessment result is a preset assessment result, the dose error during the treatment in the current radiation fraction may be determined to belong to the first type of dose error. For example, if the first assessment result is character 0, it may indicate that the first predicted radiation auxiliary image does not have similarity with the second predicted radiation auxiliary image, which may then indicate that the dose error may exist during the treatment in the current radiation fraction, and the dose error may belong to the first type of dose error caused by the positioning variation and the body posture variation of the target object.

In some embodiments, the computer device may obtain the first assessment result by comparing the first predicted radiation auxiliary image and the second predicted radiation auxiliary image using the preset assessment algorithm. If the first assessment result characterizes that the similarity between the first predicted radiation auxiliary image and the second predicted radiation auxiliary image is less than or equal to the first similarity threshold, the dose error during the treatment in the current radiation fraction may be determined to belong to the first type of dose error to characterize one or more dose errors caused by the positioning variation and the body posture variation of the target object. Therefore, the detection and identification of the first type of dose error may be realized, and accuracy of determination of the type of the dose error may be improved.

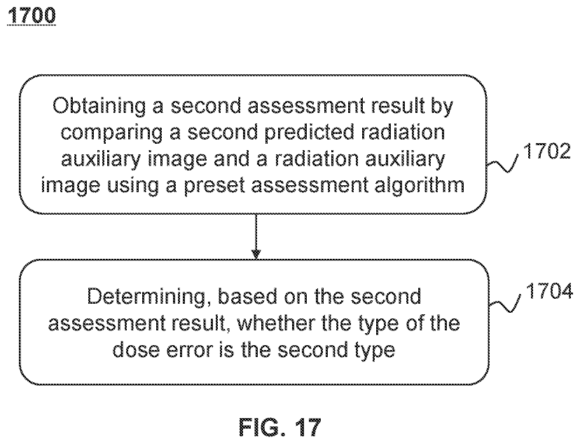

FIG. 17 is a flowchart illustrating an exemplary process for determining a second type of dose error according to some embodiments of the present disclosure. As shown in FIG. 17, process 1700 may include one or more of the following operations. In some embodiments, the process 1700 may be performed by a processing device or a system for dose verification.

In 1702, a second assessment result may be obtained by comparing a second predicted radiation auxiliary image and a radiation auxiliary image using a preset assessment algorithm. The second assessment result may be used to characterize a similarity between the second predicted radiation auxiliary image and the radiation auxiliary image.

Referring to the above embodiments, the computer device may obtain the second assessment result by performing similarity assessment using the preset assessment algorithm based on the second predicted radiation auxiliary image and the radiation auxiliary image. Descriptions regarding a manner for determining the second assessment result and the preset assessment algorithm may be found in related descriptions in the embodiments in FIG. 3, and may not be repeated herein.

In 1704, whether a dose error during treatment in a current radiation fraction belongs to a second type of dose error may be determined based on the second assessment result. The second type of the dose error may be used to characterize one or more dose errors caused by variation of one or more performance parameters of a treatment accelerator during the treatment in the current radiation fraction.

Exemplarily, if the second type of the dose error is the similarity between the second predicted radiation auxiliary image and the radiation auxiliary image, the similarity may be compared with a second similarity threshold, and if the similarity is less than or equal to the second similarity threshold, the similarity between the second predicted radiation auxiliary image and the radiation auxiliary image may be low, which in turn indicates that the second type of the dose error caused by the variation of one or more performance parameters of the treatment accelerator may exist during the treatment in the current radiation fraction, i.e., the dose error during the treatment in the current radiation fraction may be determined to belong to the second type of dose error.

On the contrary, if the second assessment result characterizes that the similarity between the second predicted radiation auxiliary image and the radiation auxiliary image is greater than the second similarity threshold, it may indicate that the second type of the dose error caused by the variation of one or more performance parameters of the treatment accelerator may not exist during the treatment in the current radiation fraction, i.e., the dose error during the treatment in the current radiation fraction may be determined not belong to the second type of dose error. Optionally, the second similarity threshold may be the same as or different from the first similarity threshold described above, which is not specifically limited in the embodiments of the present disclosure.

Certainly, the second assessment result may also be an assessment result in a form other than the similarity, such as an assessment result for characterizing whether the second predicted radiation auxiliary image is similar to or match the radiation auxiliary image, which may be represented by numbers, characters, or a combination thereof. Accordingly, a manner for determining the type of the dose error during the treatment in the current radiation fraction based on the second assessment result may also be adaptively set based on the specific form of the second assessment result.

In some embodiments, the computer device may obtain the second assessment result by comparing the second predicted radiation auxiliary image and the radiation auxiliary image using the preset assessment algorithm. If the second assessment result characterizes that the similarity between the second predicted radiation auxiliary image and the radiation auxiliary image is less than or equal to the second similarity threshold, the dose error during the treatment in the current radiation fraction may be determined to belong to the second type of the dose error used to characterize one or more dose errors caused by the variation of one or more performance parameters of the treatment accelerator. Therefore, the detection and identification of the second type of dose error may be realized, and accuracy of determination of the type of the dose error may be improved.

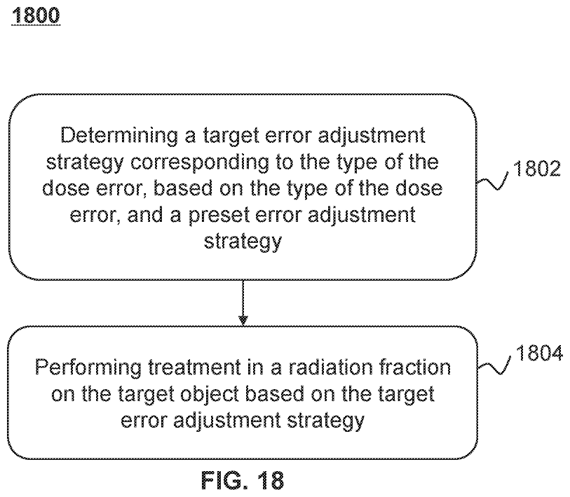

FIG. 18 is a flowchart illustrating an exemplary process for performing treatment in a radiation fraction according to some embodiments of the present disclosure. As shown in FIG. 18, process 1800 may include one or more of the following operations. In some embodiments, the process 1800 may be performed by a processing device or a system for dose verification.

In 1802, a target error adjustment strategy corresponding to the type of the dose error during the treatment in the current radiation fraction may be determined based on a type of dose error during treatment in a current radiation fraction and a preset error adjustment strategy.

The preset error adjustment strategy may include error adjustment strategies corresponding to a plurality of types of dose errors, such as a first error adjustment strategy corresponding to a first type of dose error and a second error adjustment strategy corresponding to a second type of dose error. Exemplarily, if the first type of dose error characterizes one or more dose errors caused by positioning variation and/or body posture variation of the target object, and the second type of dose error characterizes the dose error one or more dose errors caused by variation of one or more performance parameters of a treatment accelerator during the treatment in the current radiation fraction, the first error adjustment strategy may include whether to continue treatment, whether to modify a treatment plan, etc., and the second error adjustment strategy may include whether to interrupt the treatment, whether to adjust the performance parameters of the treatment accelerator, etc.

In such a case, in some embodiments, after determining the type of the dose error during the treatment in the current radiation fraction, the target error adjustment strategy corresponding to the type of the dose error during the treatment in the current radiation fraction may be determined based on the type of the dose error during the treatment in the current radiation fraction from the preset error adjustment strategy.

In 1804, the treatment in the current radiation fraction may be performed on the target object based on the target error adjustment strategy.

After determining the target error adjustment strategy, the treatment in the current radiation fraction performed on the target object, a subsequent treatment plan, or performance parameters of a treatment accelerator in a radiation device may be adjusted according to the target error adjustment strategy, to realize precise treatment in a radiation fraction on the target object.

In some embodiments, a computer device may determine, after determining the type of the dose error during the treatment in the current radiation fraction, the target error adjustment strategy corresponding to the type of the dose error during the treatment in the current radiation fraction based on the type of the dose error, and perform the treatment in the current radiation fraction on the target object based on the target error adjustment strategy. That is, in some embodiments, not only the source of the dose error may be accurately positioned, and the type of the dose error may be determined, but also a targeted dose error adjustment may be performed based on the type of the dose error. Therefore, adaptive adjustment of the treatment plan may be realized according to different states of the target object and the radiation device, precise treatment of the target object may be realized, and unnecessary radiation effects received by the target object may be reduced.

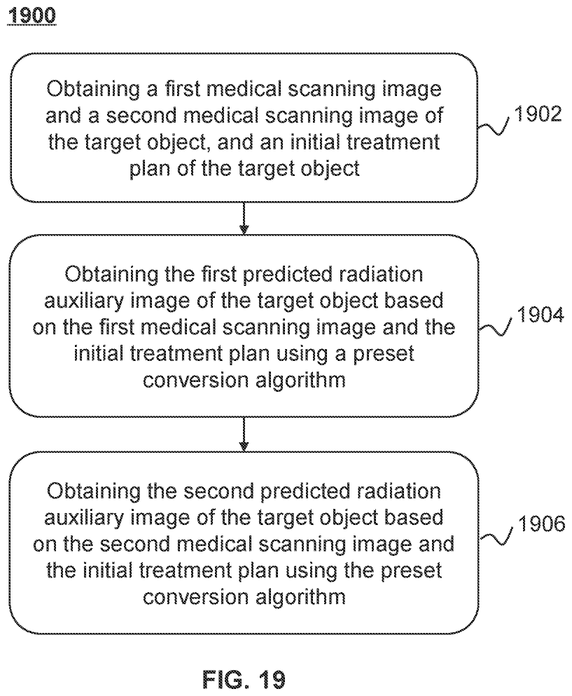

FIG. 19 is a flowchart illustrating an exemplary process for determining a second predicted radiotherapy auxiliary image according to some embodiments of the present disclosure. As shown in FIG. 19, process 1900 may include one or more of the following operations. In some embodiments, the process 1900 may be performed by a processing device or a system for dose verification.

In 1902, a first medical scanning image and a second medical scanning image of a target object during treatment in a current radiation fraction, and an initial treatment plan of the target object may be obtained.

The first medical scanning image refers to an initial medical scanning image of the target object used to make the initial treatment plan, and the second medical scanning image refers to a medical scanning image of the target object obtained before the treatment in the current radiation fraction.

That is, the initial medical scanning image of the target object obtained before the initial treatment plan is made may be the first medical scanning image, which may be used to make the initial treatment plan of the target object. The initial treatment plan may include a count of treatment in a radiation fraction and a treatment plan of treatment in each radiation fraction, which may include relevant treatment parameters corresponding to the treatment in each radiation fraction, etc. When performing the treatment in the radiation fraction on the target object based on the initial treatment plan, the medical scanning image of the target object may be obtained again and a currently obtained medical scanning image may be designated as the second medical scanning image during the treatment in the current radiation fraction. Compared with the first medical scanning image, the second medical scanning image may characterize more accurately a body posture state and a positioning state of the target object when performing the treatment in the current radiation fraction.

Since the first medical scanning image of the target object is the medical scanning image of the target object before the initial treatment plan is made, a computer device may obtain the first medical scanning image of the target object from a database or a medical image storage system. Alternatively, since the second medical scanning image of the target object is the medical scanning image of the target object obtained before the treatment in the current radiation fraction, the computer device may obtain the second medical scanning image of the target object from a medical imaging scanning device (e.g., an image scanning simulator) or an integrated radiation device with image scanning capabilities. Optionally, in the case where the medical imaging device or the integrated radiation device with image scanning capabilities sends the second medical scanning image of the target object to the database or the medical image storage system, the computer device may also obtain the second medical scanning image of the target object from the database or the medical image storage system.

Alternatively, the initial treatment plan of the target object may be obtained by the computer device from a database, a server, a local storage of the computer device, etc.

In 1904, a first predicted radiation auxiliary image of the target object during the treatment in the current radiation fraction may be obtained by using a preset conversion algorithm based on the first medical scanning image and the initial treatment plan.

In 1906, the second predicted radiation auxiliary image of the target object during the treatment in the current radiation fraction may be obtained by using the preset conversion algorithm based on the second medical scanning image and the initial treatment plan.

The preset conversion algorithm may be used to convert the medical scanning image into a predicted radiation auxiliary image, and the predicted radiation auxiliary image may be used to characterize a dose distribution of the target object under an effect of a radiation dose.

Exemplarily, when obtaining the first medical scanning image of the target object before the initial treatment plan and the second medical scanning image obtained before the treatment in the current radiation fraction, the computer device may perform image conversion by respectively inputting the first medical scanning image and the initial treatment plan, and the second medical scanning image and the initial treatment plan into the preset conversion algorithm. Thus, the first predicted radiation auxiliary image corresponding to the first medical scanning image, and the second predicted radiation auxiliary image corresponding to the second medical scanning image may be obtained. That is, the first predicted radiation auxiliary image corresponding to the initial treatment plan of the target object with an initial body posture and an initial positioning may be obtained, and the second predicted radiation auxiliary image corresponding to the treatment in the current radiation fraction of the target object with a current body posture and a current positioning may be obtained.

In the embodiment, when obtaining a predicted radiation auxiliary image of the target object at the current fraction treatment, the computer device may obtain the first medical scanning image of the target object before the initial treatment plan is made, and the second medical scanning image of the target object obtained before the treatment in the current radiation fraction, and the initial treatment plan. The first medical scanning image and the initial treatment plan, and the second medical scanning image and the initial treatment plan may be inputted into the preset conversion algorithm, respectively, to obtain the first predicted radiation auxiliary image and the second predicted radiation auxiliary image of the target object during the treatment in the current radiation fraction. That is, the first predicted radiation auxiliary image and the second predicted radiation auxiliary image of the target object during the treatment in the current radiation fraction may be obtained by using a manner of inputting the medical scanning image and the treatment plan into the preset conversion algorithm for image conversion. Thus, accuracy and efficiency of obtaining the first predicted radiation auxiliary image and the second predicted radiation auxiliary image may be improved, and efficiency of determining the type of the dose error may be improved.

In some embodiments, based on the above embodiments, a computer device may determine, based on obtaining a first medical scanning image, a second medical scanning image, and a radiation auxiliary image of a target object during treatment in a current radiation fraction, whether a total radiation dose received by the target object after the treatment in the current radiation fraction is overdosed or underdosed, which provides guidance for subsequent treatment.

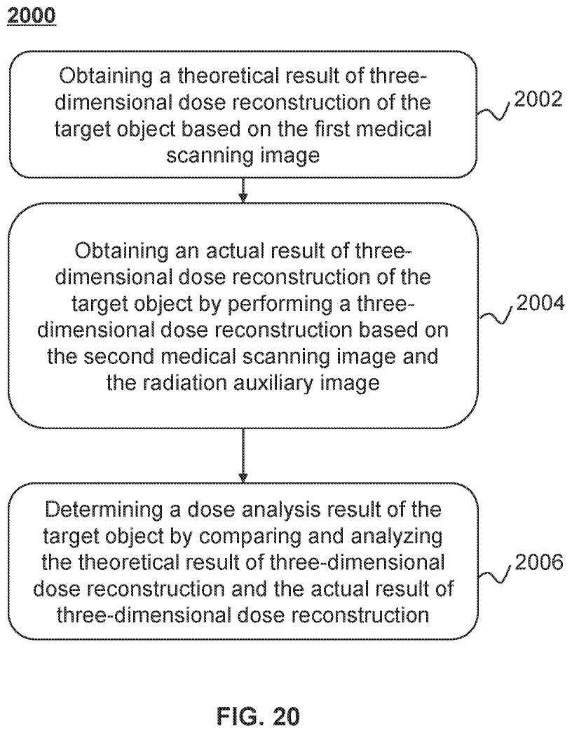

FIG. 20 is a flowchart illustrating an exemplary process for determining a dose analysis result according to some embodiments of the present disclosure. As shown in FIG. 20, process 2000 may include one or more of the following operations. In some embodiments, the process 2000 may be performed by a processing device or a system for dose verification.

In 2002, a theoretical result of three-dimensional dose reconstruction of the target object may be obtained based on the first medical scanning image.

Exemplarily, the computer device may perform the three-dimensional dose reconstruction by inputting the first medical scanning image into a preset reconstruction algorithm model to obtain the theoretical result of the three-dimensional dose reconstruction of the target object, which may be used to characterize a theoretical dose distribution received by the target object after the treatment in the current radiation fraction, the theoretical dose distribution may include theoretical dose values corresponding to different treatment portions of the target object.

In some embodiment, the preset reconstruction algorithm model may be a machine learning model, such as a Neural Network model, or an algorithm model, such as a Monte Carlo algorithm, a Fourier transform algorithm, an iterative reconstruction algorithm, or the like.

In 2004, the three-dimensional dose reconstruction may be performed based on the second medical scanning image and the radiation auxiliary image during the treatment in the current radiation fraction to obtain an actual result of the three-dimensional dose reconstruction of the target object.

Exemplarily, the computer device may input the second medical scanning image and the radiation auxiliary image obtained during the treatment in the current radiation fraction into the preset reconstruction algorithm model to perform the three-dimensional dose reconstruction to obtain the actual result three-dimensional dose reconstruction of the target object. The actual result of the three-dimensional dose reconstruction may be used to characterize the actual dose distribution received by the target object after the treatment in the current radiation fraction and may include actual dose values corresponding to different treatment portions of the target object.

In 2006, the dose analysis result of the target object may be determined.by comparing and analyzing the theoretical result of the three-dimensional dose reconstruction and the actual result of the three-dimensional dose reconstruction.

The dosage analysis result of the target object may include at least one of an underdose/overdose analysis result of a radiation dose corresponding to the different treatment portions of the target object and an underdose/overdose analysis result of a total radiation dose received by the target object.

Exemplarily, the computer device may obtain the dose analysis result corresponding to each treatment portion of the target object by comparing and analyzing the theoretical dose value of each treatment portion in the theoretical result of the three-dimensional dose reconstruction with the actual dose value of each treatment portion in the actual result of the three-dimensional dose reconstruction. A theoretical total dose value of the target object may also be determined based on the theoretical result of the three-dimensional dose reconstruction, and the actual total dose value of the target object may be determined based on the actual result of the three-dimensional dose reconstruction. A magnitude relationship between the theoretical total dose value and the actual total dose value may be judged to determine whether the total radiation dose received by the target object has the overdose/underdose analysis result.

In some embodiments, the processing device may determine a planning three-dimensional dose reconstruction result of the target object based on the initial medical scanning image of the target object.

For example, the processing device may determine the planning three-dimensional dose reconstruction result of the target object by inputting the initial medical scanning image into a preset reconstruction algorithm model.

In some embodiments, the processing device may determine an underdose/overdose analysis result for the target object by comparing at least two of the planning three-dimensional dose reconstruction result, the theoretical result of three-dimensional dose reconstruction, and the actual result of three-dimensional dose reconstruction of the target object.

In some embodiments, the processing device may determine whether a dose error exists by comparing the planning three-dimensional dose reconstruction result and the theoretical result of three-dimensional dose reconstruction, and then determine whether the dose error belongs to the first type.

In some embodiments, the processing device may determine whether a dose error exists by comparing the theoretical result of three-dimensional dose reconstruction, and the actual result of three-dimensional dose reconstruction of the target object, and then determine whether the dose error belongs to the second type.

It should be noted that the comparing operation may be performed using mathematical calculation or a model-based algorithm, which is not limited in the present disclosure.

In some embodiments, the computer device may obtain the theoretical result of the three-dimensional dose reconstruction of the target object based on the first medical scanning image and obtain the actual result of the three-dimensional dose reconstruction of the target object based on the second medical scanning image and the radiation auxiliary image of the treatment in the radiation fraction. Then, the dose analysis result of the target object may be obtained by comparing and analyzing the theoretical result of the three-dimensional dose reconstruction and the actual result of the three-dimensional dose reconstruction. The dose analysis result may be used to characterize whether the received radiation dose of the target object is overdosed or underdosed to provide data support for a subsequent treatment process, which facilitates monitoring the treatment process and adaptively adjusting a subsequent treatment plan according to an actual treatment situation, thereby improving accuracy of the treatment in the current radiation fraction.

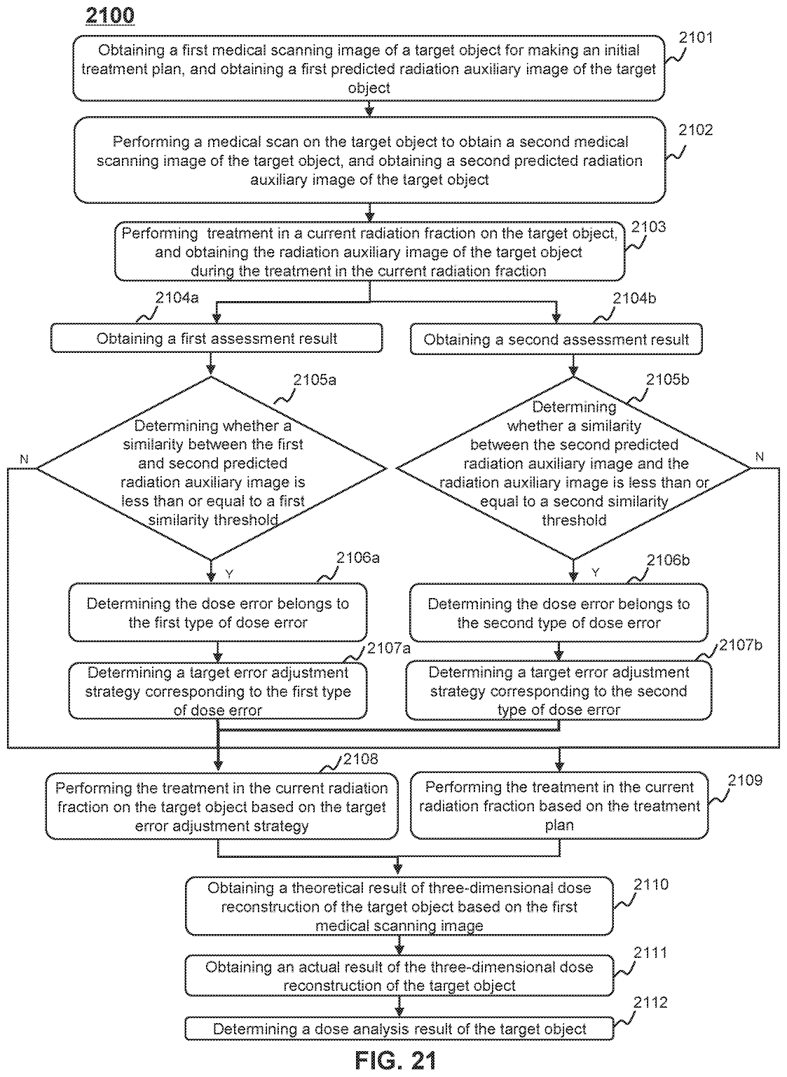

FIG. 21 is a flowchart illustrating an exemplary process for determining a type of dose error according to some embodiments of the present disclosure. As shown in FIG. 21, process 2100 may include one or more of the following operations. In some embodiments, the process 2100 may be performed by a processing device or a system for dose verification.

In 2101, a first medical scanning image of a target object for making an initial treatment plan may be obtained from a medical image storage system, and a first predicted radiation auxiliary image of a target object during the treatment in the current radiation fraction may be obtained by using a preset conversion algorithm based on the first medical scanning image and a treatment plan during the treatment in the current radiation fraction.

The treatment plan during the treatment in the current radiation fraction may be obtained from the initial treatment plan or a most recent treatment plan. The most recent treatment plan refers to a treatment plan after updating the initial treatment plan.

In 2102, an all-in-one radiation device may be controlled to perform a medical scan on the target object, a second medical scanning image of the target object may be obtained, and a second predicted radiation auxiliary image of the target object during the treatment in the current radiation fraction may be obtained by using the preset conversion algorithm based on the second medical scanning image and the treatment plan during the treatment in the current radiation fraction.

In 2103, the all-in-one radiation device may be controlled to perform the treatment in the current radiation fraction on the target object, and the radiation auxiliary image of the target object during the treatment in the current radiation fraction may be obtained.

In 2104a, a first assessment result may be obtained by comparing the first predicted radiation auxiliary image and the second predicted radiation auxiliary image using the preset assessment algorithm.

In 2105a, whether the first assessment result characterizes that a similarity between the first predicted radiation auxiliary image and the second predicted radiation auxiliary image is less than or equal to a first similarity threshold, if so, operation 2106a may be executed, if not, operation 2109 may be executed.

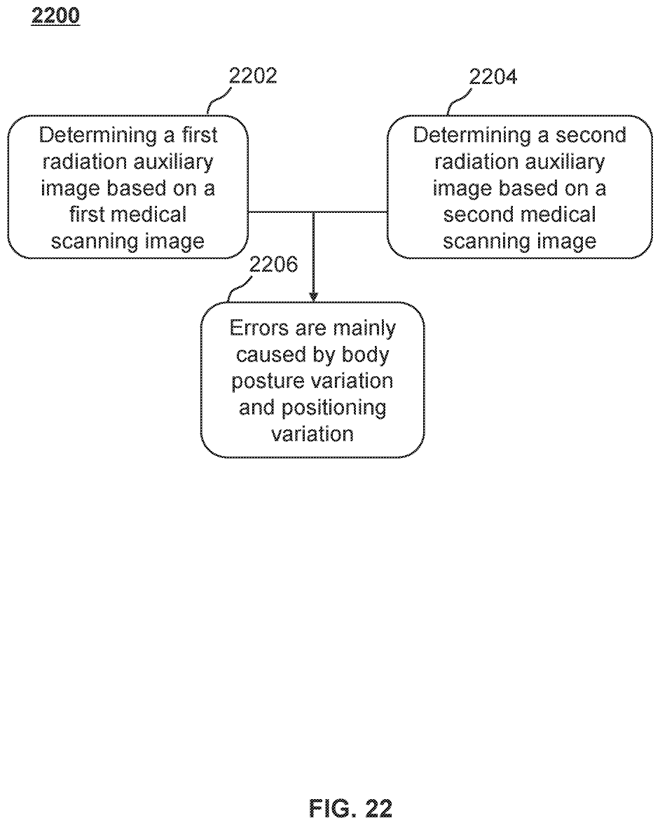

In 2106a, a dose error during the treatment in the current radiation fraction may be determined to belong to a first type of dose error, and the first type of dose error may be used to characterize one or more dose errors caused by positioning variation and body posture variation of the target object. Referring to FIG. 22.

FIG. 22 is a schematic diagram illustrating determining a type of dose error (e.g, the first type of dose error as described above) according to some embodiments of the present disclosure. As shown in FIG. 22. process 2200 may include one or more of the following operations. In some embodiments, process 2200 may be performed by the processing device or the system 100 for dose verification.

In 2202, a first radiation auxiliary image may be determined based on a first medical scanning image.

In 2204, a second radiation auxiliary image may be determined based on a second medical scanning image.

In 2206, whether error(s) are mainly caused by variation of the target object may be determined based the first medical scanning image and the second medical scanning image.

More descriptions of 2202, 2204 may be found elsewhere in the present disclosure (e.g., FIG. 15 and descriptions thereof). More descriptions of 2206 may be found elsewhere in the present disclosure (e.g., FIGS. 15, 19 and descriptions thereof).

In 2107a, a target error adjustment strategy corresponding to the first type of the dose error may be determined from a preset error adjustment strategy. Then operation 2108 may be performed.

In 2104b, a second assessment result may be obtained by comparing the second predicted radiation auxiliary image and the radiation auxiliary image using the preset assessment algorithm.

In 2105b, whether the second assessment result characterizes that a similarity between the second predicted radiation auxiliary image and the radiation auxiliary image is less than or equal to a second similarity threshold may be determined. If so, operation 2106b may be executed, if not, operation 2109 may be executed.

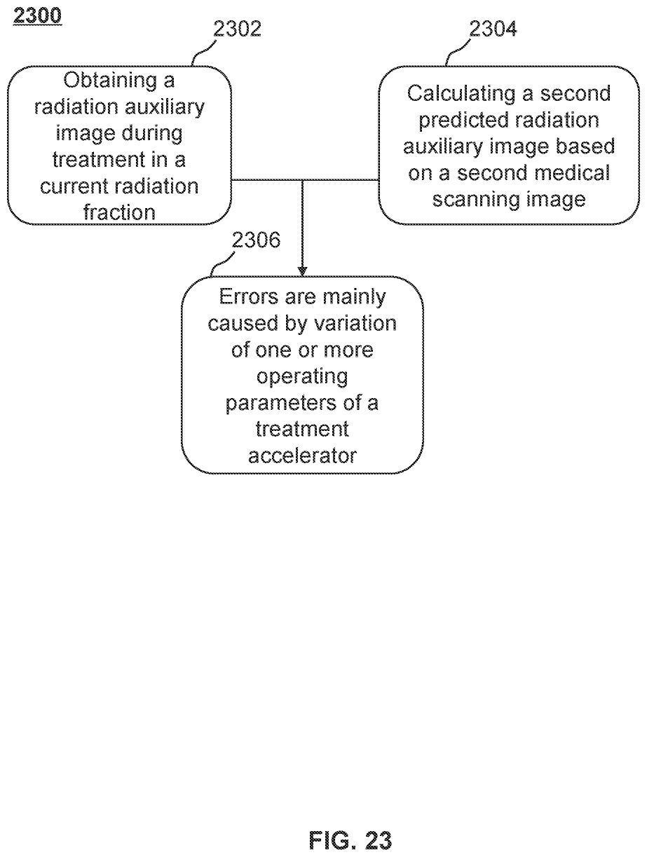

Step 2106b, the dose error during the treatment in the current radiation fraction may be determined to belong to a second type of dose error, and the second type of dose error may be used to characterize one or more dose errors caused by variation of one or more performance parameters of a treatment accelerator during the treatment in the current radiation fraction. Referring to FIG. 23.

FIG. 23 is a schematic diagram illustrating determining a type of dose error (e.g, the first type of dose error as described above) according to some embodiments of the present disclosure. As shown in FIG. 23. process 2300 may include one or more of the following operations. In some embodiments, process 2300 may be performed by the processing device or the system 100 for dose verification.

In 2302, a radiation auxiliary image may be obtained during treatment in a current radiation fraction.

In 2304, a second predicted radiation auxiliary image may be calculated based on a second medical scanning image.

In 2306, whether error(s) are mainly caused by variation of parameters of the treatment accelerator may be determined based the radiation auxiliary image and the second predicted radiation auxiliary image.

More descriptions may be found elsewhere in the present disclosure (e.g., FIG. 17 and descriptions thereof).

In 2107b, a target error adjustment strategy corresponding to the second type of dose error may be determined from the preset error adjustment strategy. Then operation 2108 may be executed.

In 2108, the treatment in the current radiation fraction may be performed on the target object based on the target error adjustment strategy. Then operation 2109 may be executed.

In 2109, the treatment in the current radiation fraction may be performed based on the treatment plan during the treatment in the current radiation fraction. Then operation 2110 may be performed.

In 2110, a theoretical result of three-dimensional dose reconstruction of the target object may be obtained based on the first medical scanning image.

In 2111, an actual result of the three-dimensional dose reconstruction of the target object may be obtained by performing the three-dimensional dose reconstruction based on the second medical scanning image and the radiation auxiliary image during the treatment in the current radiation fraction.

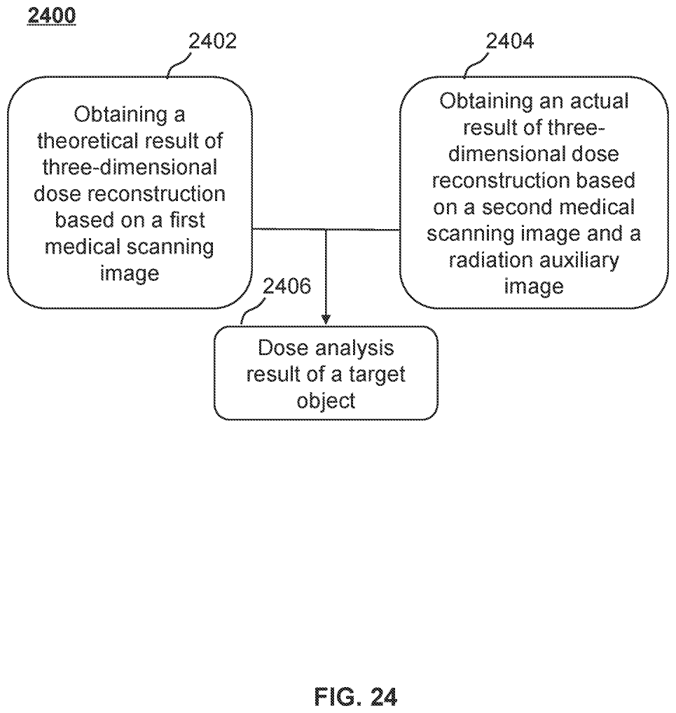

In 2112, a dose analysis result of the target object may be determined by comparing and analyzing the theoretical result of the three-dimensional dose reconstruction and the actual result of the three-dimensional dose reconstruction. Referring to FIG. 24.

FIG. 24 is a schematic diagram illustrating dose analysis of treatment in a radiation fraction according to some embodiments of the present disclosure. As shown in FIG. 24. process 2400 may include one or more of the following operations. In some embodiments, process 2400 may be performed by the processing device or the system 100 for dose verification.

In 2402, a theoretical result of three-dimensional dose reconstruction may be obtained based on a first medical scanning image.

In 2404, an actual result of three-dimensional dose reconstruction may be obtained based on a second medical scanning image and a radiation auxiliary image.

In 2406, a dose analysis result of a target object may be determined based on the theoretical result of three-dimensional dose reconstruction and the actual result of three-dimensional dose reconstruction.

More descriptions may be found elsewhere in the present disclosure (e.g., FIG. 20 and descriptions thereof).

It should be noted that the above description of process 2100 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, only the operation(s) relating to the first type of dose error may be performed. As another example, only the operation(s) relating to the second type of dose error may be performed. As another example, different embodiments in the process 2100 may be performed separately or independently (e.g., the determination of the first type and the second type of dose error may be performed in different time periods).

In some embodiments, a computer device may determine a plurality of types of dose errors of the target object based on predicted EPID images and actual EPID images obtained under different states. Since the predicted EPID images and the actual EPID images of different states may characterize different states of the target object and different states of a radiation device, factors causing the dose errors may be accurately determined, i.e., a source of the dose errors may be determined to obtain the type of the dose error, thereby improving accuracy and reasonableness of determining the type of the dose error. In addition, since the method based on the present application is capable of accurately locating the source of the dose error, the source of the dose error can also be adjusted in a timely manner when the source of the dose error has been determined, thereby, reducing the error and improve the precision of the fractionated treatment.

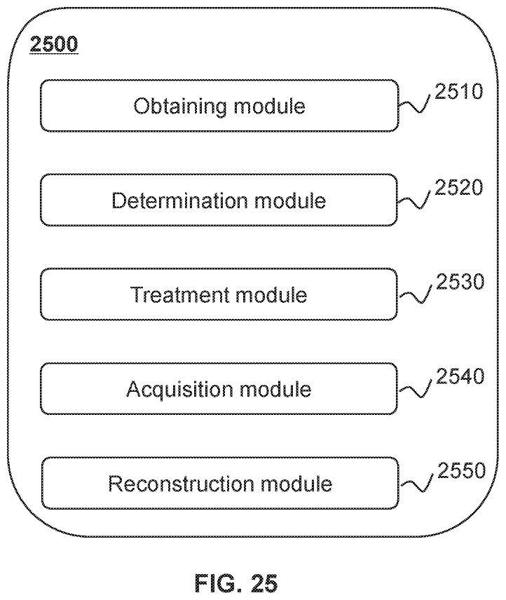

FIG. 25 is an exemplary diagram illustrating modules of a dose verification system according to some embodiments of the present disclosure. As shown in FIG. 25, system 2500 may include an acquisition module 2510, a determination module 2520, a treatment module 2530, a collection module 2540, and a reconstruction module 2550.

The acquisition module 2510 may be configured to obtain a predicted radiation auxiliary image of a target object at a target radiation time point or a current radiation fraction.

The determination module 2520 may be configured to determine a target dose strategy based on the predicted radiation auxiliary image.

The treatment module 2530 may be configured to perform treatment in a current radiation fraction on the target object based on the target dose strategy.

The collection module 2540 may be configured to obtain a radiation auxiliary image at the target radiation time point or a current radiation fraction.

The reconstruction module 2550 may be configured to reconstruct a radiation dose of the target radiation time point or the current radiation fraction based on the radiation auxiliary image.

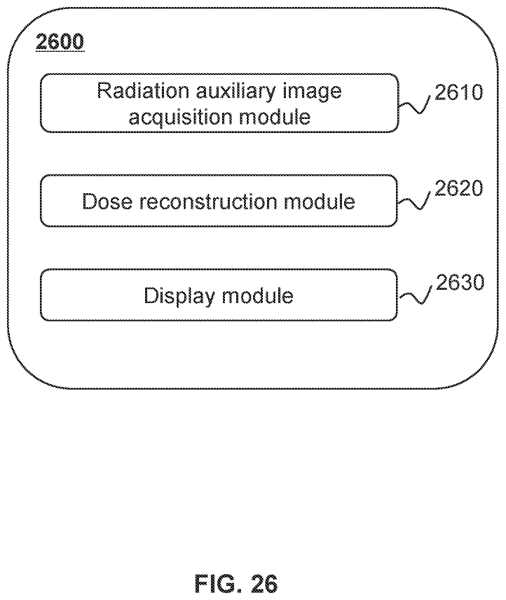

FIG. 26 is an exemplary diagram illustrating modules of a system for online radiation dose reconstruction according to some embodiments of the present disclosure. As shown in FIG. 26, a system 2600 may include a radiation auxiliary image acquisition module 2610, a dose reconstruction module 2620, and a real-time display module 2630.

The radiation auxiliary image acquisition module 2610 may be configured to obtain in real-time a radiation auxiliary image corresponding to a current radiation field in a plurality of radiation fields in a current treatment process for a target object.

The dose reconstruction module 2620 may be configured to reconstruct a radiation dose in real-time based on the radiation auxiliary image corresponding to the current radiation field.

The real-time display module 2630 may be configured to display in real-time a radiation dose corresponding to the current radiation field in the current treatment process or display in real-time a cumulative result of radiation doses corresponding to the plurality of radiation fields in the current treatment process.

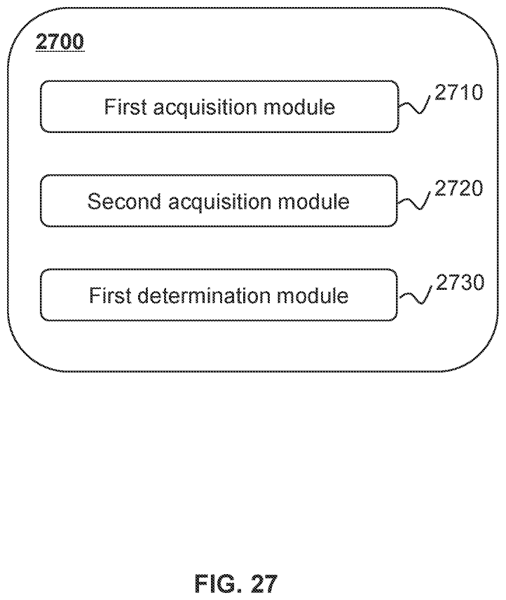

FIG. 27 is an exemplary diagram illustrating modules of a system for determining a type of dose error according to some embodiments of the present disclosure. As shown in FIG. 27, system 2700 may include a first acquisition module 2710, a second acquisition module 2720, and a first determination module 2730.

The first acquisition module 2710 may be configured to obtain a predicted radiation auxiliary image of a target object at a target radiation time point or a current radiation fraction. The predicted radiation auxiliary image may include a first predicted radiation auxiliary image and a second predicted radiation auxiliary image. The first predicted radiation auxiliary image may be obtained based on an initial medical scanning image of the target object, and the second predicted radiation auxiliary image may be obtained based on a medical scanning image of the target object at the target radiation time point or the current radiation fraction.

The second acquisition module 2720 may be configured to obtain the radiation auxiliary image of the target object at the target radiation time point or the current radiation fraction.

The first determination module 2730 may be configured to determine a type of dose error of the current radiation fraction based on the first predicted radiation auxiliary image, the second predicted radiation auxiliary image, and the radiation auxiliary image.

The embodiments of the present disclosure may include but are not limited to the following beneficial effects. (1) Real-time reconstruction may be performed on a radiation dose by obtaining a radiation auxiliary image corresponding to a current radiation field in a plurality of radiation fields in a current treatment process, so that a radiation dose may be fed back in real-time to a doctor, the doctor can optimize a subsequent treatment plan based on a real-time displayed radiation dose to treat a patient better. (2) The radiation dose may be displayed in real-time, which enables the doctor to be more proactive in subsequent operations when the radiation dose in the current treatment process is viewed on time. (3) The current radiation field or a result of the current treatment process may be evaluated by combining a two-dimensional pass rate matrix, the doctor may intuitively understand a treatment effect of the current radiation field or the current treatment process, which facilitates performing adjustments to the subsequent treatment plan, thereby treating the patient better.

It should be noted that different embodiments may produce different beneficial effects, and in different embodiments, the beneficial effects that may be produced may be any one or a combination of any one or more of the above, or any other beneficial effect that may be obtained.

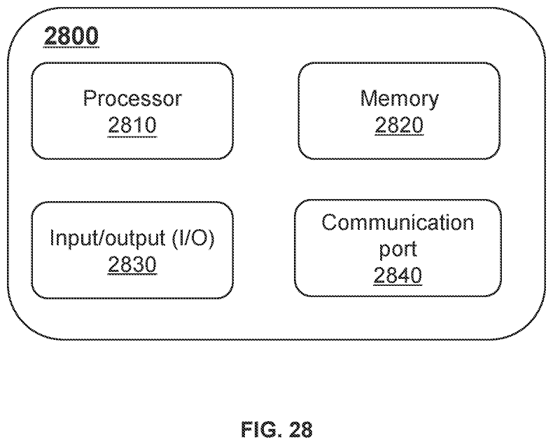

FIG. 28 is a schematic diagram illustrating an exemplary hardware and/or software components of a computing device according to some embodiments of the present disclosure.

A computing device 2800 may include a processor 2810, a memory 2820, an input/output (I/O) 2830, and a communication port 2840.

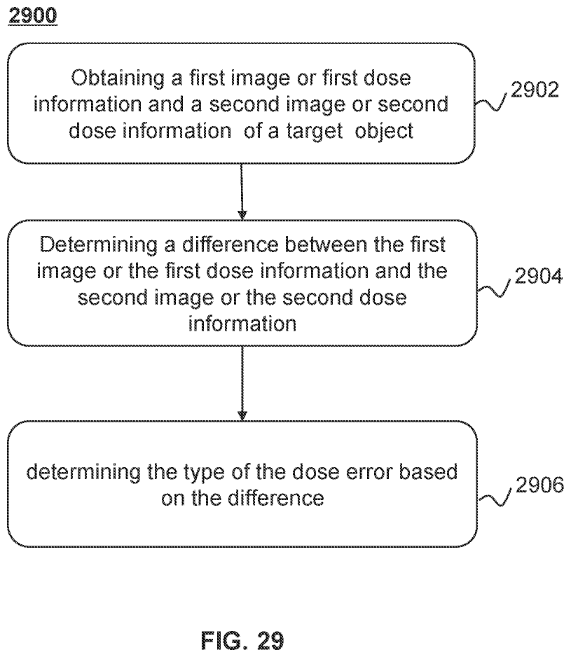

FIG. 29 is a flowchart illustrating an exemplary process for determining a dose error according to some embodiments of the present disclosure. As shown in FIG. 29, process 2900 may include one or more of the following operations. In some embodiments, the process 2900 may be performed by a processing device or a system for dose verification.

In 2902, obtaining a first image or first dose information and a second image or second dose information of a target object. In an embodiment, the first image may be the first predicted radiation auxiliary image, and the second image may be the second predicted radiation auxiliary image of the target. In another embodiment, the first image may be the second predicted radiation auxiliary image, and the second image may be the radiation auxiliary image of the target. In yet another embodiment, the first dose information may be a theoretical result of three-dimensional dose reconstruction based on a first medical scanning image, and the second image may be an actual result of three-dimensional dose reconstruction based on a second medical scanning image and a radiation auxiliary image.

In 2904, determining a difference between the first image or the first dose information and the second image or the second dose information. For example, the difference may be the variation between the first and second images or the dose offset at one or more positions between the first and second dose information.

In 2906, determining the type of the dose error based on the difference. For example, the type of the dose error may be determined as the errors caused by body posture variation and positioning variation based on the difference between the first predicted radiation auxiliary image and the second predicted radiation auxiliary image of the target.

In some embodiments, the first image may be a medical scanning image obtained before performing treatment at a current radiation fraction on the target object, such as a CT image, an MR image, or the like. At this time, the first image may also be referred to as an initial medical scanning image or a first medical scanning image. The second image may be a medical scanning image obtained by a treatment device emitting radiation beam(s) during the treatment at the current radiation fraction of the target object, such as a CT image or an MR image. At this time, the second image may also be referred to as a second medical scanning image. The difference between the first image and the second image may be determined by further processing the first image and the second image. For example, a first predicted radiation auxiliary image may be obtained based on the first image, and a second predicted radiation image may be obtained based on the second image. The first predicted radiation auxiliary image and the second predicted radiation auxiliary image may be compared to determine the difference.

In some embodiments, the first image may be the medical scanning image obtained by the treatment device emitting the radiation beam(s) during the treatment at the current radiation fraction of the target object. The second image may be the medical scanning image obtained before performing treatment at the current radiation fraction on the target object. A processing device may obtain the second predicted radiation auxiliary image based on the first predicted radiation auxiliary image and determine the difference based on the second predicted radiation auxiliary image and the second image.

In some embodiments, the first image may be the first predicted radiation auxiliary image, and the second image may be the second predicted radiation auxiliary image. The processing device may determine the difference by directly performing comparative analysis on the first image and the second image.

In some embodiments, the first image may be the second predicted radiation auxiliary image, and the second image may be a radiation auxiliary image (the actual radiation auxiliary image). The processing device may determine the difference by directly performing comparative analysis on the first image and the second image.

It should be noted that the first image and the second image are partially illustrated hereinabove. The first image may be the initial medical scanning image, the second medical scanning image, the first predicted radiation auxiliary image, or the second predicted radiation auxiliary image. The second image may be the second medical scanning image, the second predicted radiation auxiliary image, or the radiation auxiliary image. Different types of error may be determined based on the comparative analysis on different combinations of the first image and the second image.

In some embodiments, the first image may be pre-scanned and stored in a storage device or an imaging device, and the processing device may obtain the first image by reading from the storage device or the imaging device. The second image may be obtained by scanning the target object using the imaging device (e.g., a CT device or an MR device) during the treatment at the current radiation fraction.

In some embodiments, the first dose information refers to a planning three-dimensional dose reconstruction result determined by calculating based on the first image and a treatment plan of the target object. The planning three-dimensional dose reconstruction result may be used to characterize a planning dose distribution that the target object should receive after receiving treatment at a radiation fraction according to the treatment plan. A planning distribution may include planning dose values corresponding to different treatment parts of the target object. The first dose information may be obtained by three-dimensional dose reconstruction based on inputting the first medical scanning image into a preset reconstruction algorithm model.

In some embodiments, the second dose information refers to a theoretical result of three-dimensional dose reconstruction obtained by performing dose reconstruction based on the second image. The theoretical result of three-dimensional dose reconstruction may be used to characterize a theoretical dose distribution received by the target object after the treatment at the current radiation fraction. The theoretical dose distribution may include theoretical dose values corresponding to different treatment parts of the target object. The second dose information may be obtained by the three-dimensional dose reconstruction based on inputting the first medical scanning image into a preset reconstruction algorithm model.

In some embodiments, the first dose information may also be the above-mentioned theoretical result of the three-dimensional dose reconstruction. The second dose information may be an actual result of the three-dimensional dose reconstruction obtained by reconstructing the medical scanning image (the second medical scanning image) collected in real time during the treatment at the current radiation fraction. The processing device may input the second medical scanning image and the radiation auxiliary images collected during treatment at the current radiation fraction into the preset reconstruction algorithm model for three-dimensional dose reconstruction to obtain an actual result of the three-dimensional dose reconstruction. The actual result of the three-dimensional dose reconstruction may be used to characterize an actual dose distribution received by the target object after the treatment at the current radiation fraction. The actual dose distribution may include actual dose values corresponding to different treatment parts of the target object.

The preset reconstruction algorithm model may be a machine learning model, such as a Neural Network model, or an algorithm model, such as a Monte Carlo algorithm, a Fourier transform algorithm, an iterative reconstruction algorithm, or the like.

The difference may be a pixel difference between the first image and the second image, or a dose distribution difference between the first dose information and the second dose information. In some embodiments, the processing device may determine the dose distribution difference by performing comparative analysis on the first image and the second image, or by performing comparative analysis on the first dose information and the second dose information.

The type of dose error may include whether there is a dose error, errors caused by changes in the body of the patient and errors caused by a radiation device.

More descriptions regarding the operations of FIG. 29 may be found elsewhere in the present disclosure. For example, related descriptions in FIG. 15-FIG. 24, which may not be repeated here.

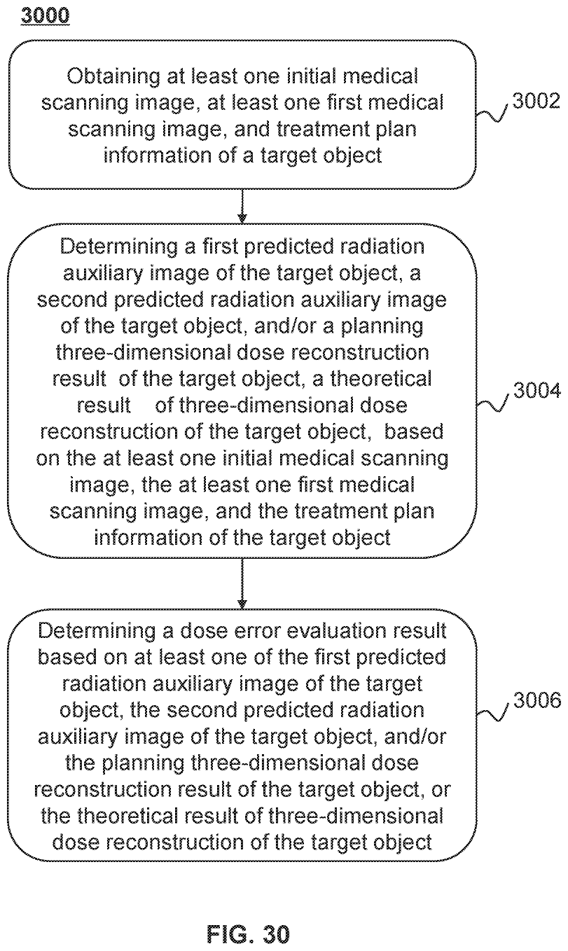

FIG. 30 is a flowchart illustrating an exemplary process of a method for dose error evaluation according to some embodiments of the present disclosure. As shown in FIG. 30, process 3000 may include one or more of the following operations. In some embodiments, the process 3000 may be performed by a processing device or a system for dose verification.

In 3002, obtaining at least one initial medical scanning image, at least one first medical scanning image, and treatment plan information of a target object;

In 3004, determining a first predicted radiation auxiliary image of the target object, a second predicted radiation auxiliary image of the target object, and/or a planning threc-dimensional dose reconstruction result of the target object, a theoretical result of three-dimensional dose reconstruction of the target object, based on the at least one initial medical scanning image, the at least one first medical scanning image, and the treatment plan information of the target object;

In 3006, determining a dose error evaluation result based on at least one of the first predicted radiation auxiliary image of the target object, the second predicted radiation auxiliary image of the target object, and/or the planning three-dimensional dose reconstruction result of the target object, or the theoretical result of three-dimensional dose reconstruction of the target object.

In some embodiments, the dose error evaluation result may include whether a dose error exists, whether the dose error is caused by variation of the target object, whether the dose error is caused by a treatment device (e.g., a treatment accelerator) of the target object.

In some embodiments, the dose error caused by variation of the target object may also be referred to as dose error with the first type. The dose error caused by the treatment device may also be referred to as dose error with the second type.

More descriptions of the dose error evaluation may be found elsewhere in the present disclosure. For example, more descriptions of the obtaining at least one initial medical scanning image and at least one first medical scanning image may be found in FIG. 19 and related descriptions thereof; more descriptions of the obtaining predicted radiation auxiliary image(s) and three-dimensional dose reconstruction result(s) may be found in FIGS. 19-20 and related descriptions thereof; more descriptions of the determining the dose error evaluation result may be found in FIGS. 2, 15-19 and related descriptions thereof.

The processor 2810 may execute computer instructions (e.g., program code) and perform functions of the processing device 130 according to the method(s) described herein. The computer instructions may include, for example, routines, programs, objects, components, data structures, procedures, modules, and functions, which perform particular functions described herein. For example, the processor 2810 may process data of the radiation device 110, the terminal 140, the storage device 150, and/or any other component in the system 100. In some embodiments, the processor 2810 may include at least one hardware processor, such as a microcontroller, microprocessor, reduced instruction set computer (RISC), application specific integrated circuit (ASIC), application specific instruction set processor (ASIP), central processing unit (CPU), graphics processing unit (GPU), physical processing unit (PPU), microcontroller unit, digital signal processor (DSP), field programmable gate array (FPGA), high-order RISC Machine (ARM), programmable logic device (PLD), any circuit or processor or similar capable of performing at least one function, or any combination thereof.

Merely for illustration, only one processor is described in the computing device 2800. However, it should be noted that the computing device 2800 in the present disclosure may also include multiple processors, thus operations and/or method steps that are performed by one processor as described in the present disclosure may also be jointly or separately performed by the multiple processors. For example, if in the present disclosure the processor of the computing device 2800 executes both operations A and B, it should be understood that operation A and operation B may also be performed by two or more different processors jointly or separately in the computing device 2800 (e.g., a first processor executes operation A and a second processor executes operation B, or the first and second processors jointly execute operations A and B).

The memory 2820 may store data/information obtained from the radiation device 110, the terminal 140, the storage device 150, and/or any other component in the system 100. In some embodiments, the memory 2820 may include a mass storage, a removable storage, a volatile read-write memory, a read-only memory (ROM), or any combination thereof. For example, the mass storage may include a magnetic disk, an optical disk, a solid-state hard disk, or the like. The removable memory may include a flash drive, a floppy disk, an optical disk, a memory card, a compressed disk, a magnetic tape, etc. The volatile read and write memory may include a random access memory (RAM). The RAM may include a dynamic RAM (DRAM), a double rate synchronous dynamic RAM (DDRSDRAM), a static RAM (SRAM), a thyristor RAM (t-ram), a zero capacitance (Z-RAM), etc. The exemplary read-only memory may include a masked read-only memory (MROM), a programmable read-only memory (PROM), an erasable programmable read-only memory (EPROM), an electrically erasable programmable read-only memory (EEPROM), an optical disk read-only memory (CD-ROM), a digital multifunctional disk read-only memory, or the like. In some embodiments, the memory 2820 may store at least one program and/or instruction for executing the exemplary manner described in the present disclosure.

The input/output (I/O) 2830 may be used to input and/or output signal, data, information, etc. In some embodiments, the input/output (I/O) 2830 may enable the user to interact with processing device 130. In some embodiments, the input/output (I/O) 2830 may include an input device and an output device. An exemplary input device may include a keyboard, a mouse, a touch screen, a microphone, or any combination thereof. The exemplary output device may include a display device, a speaker, a printer, a projector, or any combination thereof. An exemplary display device may include a liquid crystal display (LCD), a light emitting diode (LED)-based display, a flat panel display, a curved surface display, a television device, a cathode ray tube, or any combination thereof.

The communication port 2840 may be connected with a network (e.g., the network 120) to facilitate data communication. The communication port 2840 may establish a connection between the processing device 130 and the radiation device 110, the terminal 140, and/or the storage device 150. The connection may include a wired connection and a wireless connection. The wired connection may include, for example, cable, optical cable, telephone line, or any combination thereof. The wireless connection may include, for example, Bluetooth link, Wi-Fi™ link, WiMax™ link, WLAN link, ZigBee link, mobile network link (e.g., 3G, 4G, 5g, etc.), or any combination thereof. In some embodiments, the communication port 2840 may be and/or include a standardized communication port, such as RS232, RS485, etc. In some embodiments, the communication port 2840 may be a specially designed communication port. For example, the communication port 2840 may be designed according to a digital imaging and medical communication (DICOM) protocol.

The basic concepts have been described. Obviously, for those skilled in the art, the detailed disclosure may be only an example and may not constitute a limitation to the present disclosure. Although not explicitly stated here, those skilled in the art may make various modifications, improvements, and amendments to the present disclosure. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of the specification are not necessarily all referring to the same embodiment. In addition, some features, structures, or features in the present disclosure of one or more embodiments may be appropriately combined.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, all aspects of the present disclosure may be performed entirely by hardware, may be performed entirely by software (including firmware, resident software, microcode, etc.), or may be performed by a combination of hardware and software. The above hardware or software may be referred to as “data block”, “module”, “engine”, “unit”, “component”. or “system”. In addition, aspects of the present disclosure may appear as a computer product located in one or more computer-readable media, the product including computer-readable program code.

A computer storage medium may include a propagation data signal containing a computer program encoding, such as on a baseband or as part of a carrier. The propagation signal may have a variety of expressions, including an electromagnetic form, an optical form, or a suitable combination form. The computer storage medium may be any computer-readable medium other than the computer-readable storage medium, which may be used to perform system, devices, or devices to implement communication, propagating, or devices by connecting to an instruction. The program code located on the computer storage medium may be propagated through any suitable medium, including radio, cable, fiber optic cable, RF, or similar media, or any combination of the foregoing.

Computer program code for carrying out operations for aspects of the present disclosure may be written in any combination of one or more programming languages, including an object- oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter case, the remote computer may be connected to the user's computer through any network, such as a local area network (LAN) or a wide area network (WAN), or connected to an external computer (e.g., via the Internet), or in a cloud computing environment, or as a service use such as software as a service (Saas).

Moreover, unless otherwise specified in the claims, the sequence of the processing elements and sequences of the present application, the use of digital letters, or other names are not used to define the order of the application flow and methods. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various assemblies described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure and aiding in the understanding of one or more of the various embodiments. However, this disclosure may not mean that the present disclosure object requires more features than the features mentioned in the claims. In fact, the features of the embodiments are less than all of the features of the individual embodiments disclosed above.

In some embodiments, the numbers expressing quantities, properties, and so forth, used to describe and claim certain embodiments of the application are to be understood as being modified in some instances by the term “about,” “approximate,” or “substantially.” Unless otherwise stated, “about,” “approximate,” or “substantially” may indicate a +20% variation of the value it describes. Accordingly, in some embodiments, the numerical parameters set forth in the description and attached claims are approximations that may vary depending upon the desired properties sought to be obtained by a particular embodiment. In some embodiments, the numerical parameters should be construed in light of the number of reported significant digits and by applying ordinary rounding techniques. Although the numerical domains and parameters used in the present application are used to confirm the range of ranges, the settings of this type are as accurate in the feasible range in the feasible range in the specific embodiments.

Each patent, patent application, patent application publication, and other materials cited herein, such as articles, books, instructions, publications, documents, etc., are hereby incorporated by reference in the entirety. In addition to the application history documents that are inconsistent or conflicting with the contents of the present disclosure, the documents that may limit the widest range of the claim of the present disclosure (currently or later attached to this application) are excluded from the present disclosure. It should be noted that if the description, definition, and/or terms used in the appended application of the present disclosure is inconsistent or conflicting with the content described in the present disclosure, the use of the description, definition and/or terms of the present disclosure shall prevail.

At last, it should be understood that the embodiments described in the disclosure are used only to illustrate the principles of the embodiments of this application. Other modifications may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for dose verification implemented on a computing device having one or more processors and one or more storage devices, comprising:
obtaining a predicted radiation auxiliary image of a target object at a target radiation time point;
determining a target dose strategy based on the predicted radiation auxiliary image; and
performing, based on the target dose strategy, treatment in a current radiation fraction on the target object.

2. The method of claim 1, further comprising:
obtaining a radiation auxiliary image of the target object at the target radiation time point; and
reconstructing, based on the radiation auxiliary image, a radiation dose at the target radiation time point.

3. The method of claim 2, wherein the reconstructing a radiation dose at the target radiation time point includes:
obtaining an initial fluence map corresponding to the target radiation time point and data related to a radiation source of the treatment;
determining a target fluence map corresponding to the target radiation time point based on at least the radiation auxiliary image, the initial fluence map, and the data related to the radiation source;
obtaining a target scanning image of the target object; and
determining the radiation dose received by the target object at the target radiation time point based on the target fluence map, the target scanning image, and the data related to the radiation source.

4. The method of claim 3, wherein the determining a target fluence map corresponding to the target radiation time point includes determining the target fluence map corresponding to the target radiation time point through one or more iterations, and a current iteration of the one or more iterations includes:
obtaining object information of the target object;
determining, based on the data related to the radiation source, a current fluence map corresponding to the current iteration, and the object information, a prediction image of radiation in the current iteration, wherein the initial fluence map is designated as a current fluence map corresponding to a first iteration of the one or more iterations;
determining whether the radiation auxiliary image and the prediction image of radiation in the current iteration satisfy a first judgment condition;
in response to the radiation auxiliary image and the prediction image of radiation in the current iteration satisfying the first judgment condition, designating the current fluence map corresponding to the current iteration as the target fluence map; and
in response to the radiation auxiliary image and the prediction image of radiation in the current iteration not satisfying the first judgment condition, updating the current fluence map corresponding to the current iteration, and designating the updated current fluence map corresponding to the current iteration as a current fluence map corresponding to a next iteration of the one or more iterations.

5. The method of claim 3, wherein the determining a target fluence map corresponding to the target radiation time point includes determining the target fluence map corresponding to the target radiation time point through one or more iterations, and a current iteration of the one or more iterations includes:
obtaining object information of the target object;
determining a prediction image of main radiation beam and a scattering ratio in the current iteration based on the data related to the radiation source, a current fluence map corresponding to the current iteration, and the object information, wherein the initial fluence map is designated as a current fluence map corresponding to a first iteration of the one or more iterations;
determining a de-scattering reference image in the current iteration based on the scattering ratio and the radiation auxiliary image;
determining whether the de-scattering reference image and the prediction image of main radiation beam in the current iteration satisfy a second judgment condition;
in response to the de-scattering reference image and the prediction image of main radiation beam in the current iteration satisfying the second judgment condition, designating the current fluence map corresponding to the current iteration as the target fluence map; and
in response to the de-scattering reference image and the prediction image of main radiation beam in the current iteration not satisfying the second judgment condition, updating the current fluence map corresponding to the current iteration, and designating the updated current fluence map corresponding to the current iteration as a current fluence map corresponding to a next iteration.

6. The method of claim 3, wherein the obtaining a target scanning image of the target object includes:
obtaining a plurality of scanning images of the target object, the plurality of scanning images including a sequence of images corresponding to a plurality of phases respectively; and
determining the target scanning image from the plurality of scanning images based on the radiation auxiliary image.

7. The method of claim 6, wherein the determining the target scanning image from the plurality of scanning images based on the radiation auxiliary image includes:
determining, from the plurality of scanning images, a plurality of prediction phase images of the target object at the target radiation time point corresponding to the plurality of phases, respectively;
determining, from the plurality of prediction phase images, a matched image that matches the radiation auxiliary image; and
determining a target phase corresponding to the matched image; and
designating a scanning image corresponding to the target phase as the target scanning image.

8. The method of claim 7, wherein the determining a plurality of prediction phase images corresponding to the plurality of phases, respectively, includes:
obtaining treatment planning information;
determining planning delivery information at the target radiation time point based on the treatment planning information, wherein the planning delivery information includes one or more radiation beam angles and a segment parameter corresponding to each of the one or more radiation beam angles; and
for each phase of the plurality of phases, determining a prediction phase image corresponding to the phase based on the planning delivery information and a scanning image corresponding to the phase.

9. The method of claim 2, wherein the reconstructing a radiation dose at the target radiation time point includes:
obtaining in real-time a radiation auxiliary image corresponding to each current radiation field in a plurality of radiation fields in a current treatment process for the target object;
reconstructing in real-time the radiation dose at the target radiation time point based on the radiation auxiliary image corresponding to the current radiation field; and
displaying in real-time the radiation dose corresponding to the current radiation field in the current treatment process, or displaying in real-time a cumulative result of radiation doses corresponding to the plurality of radiation fields in the current treatment process.

10. The method of claim 9, further comprising:
determining a comparison result by comparing a real-time reconstructed radiation dose with an expected dose.

11. The method of claim 10, further comprising:
providing in real-time an underdose/overdose analysis result for a radiation target region or an organ at risk based on the comparison result.

12. The method of claim 9, wherein the reconstructing in real-time the radiation dose at the target radiation time point based on the radiation auxiliary image corresponding to the current radiation field includes:
in response to a completion of obtaining the radiation auxiliary image corresponding to the current radiation field, automatically reconstructing the radiation dose in real-time based on the radiation auxiliary image corresponding to the current radiation field.

13. The method of claim 9, further comprising:
sending the radiation auxiliary image to a radiotherapy planning system to facilitate real-time reconstructing of the radiation dose in the radiotherapy planning system; and
displaying in real-time a reconstruction result of the radiation dose using a terminal device in communication with the radiotherapy planning system.

14. The method of claim 9, further comprising:
obtaining a two-dimensional pass rate matrix based on the radiation auxiliary image; and
determining an evaluation result of the current radiation field or the current treatment process based on the two-dimensional pass rate matrix, and at least one of the radiation dose corresponding to the current radiation field or the cumulative result of radiation doses in the current treatment process.

15. The method of claim 1. further comprising:
obtaining a first image or first dose information and a second image or second dose information of a target object;
determining a difference between the first image or the first dose information and the second image or the second dose information; and
determining the type of the dose error based on the difference.

16. The method of claim 15, wherein the determining a target dose strategy based on the predicted radiation auxiliary image includes:
determining whether a first type of dose error exists based on the predicted radiation auxiliary image; and
determining the target dose strategy based on a determination result of whether the first type of dose error exists.

17. The method of claim 16, wherein the predicted radiation auxiliary image includes a first predicted radiation auxiliary image and a second predicted radiation auxiliary image, and the determining whether a first type of dose error exists based on the predicted radiation auxiliary image includes:
obtaining a first assessment result by comparing the first predicted radiation auxiliary image and the second predicted radiation auxiliary image using a preset assessment algorithm; and
determining, based on the first assessment result, whether a dose error at the target radiation time point belongs to the first type of dose error, the first type of dose error being configured to characterize one or more dose errors caused by positioning variation and/or body posture variation of the target object.

18. The method of claim 15, wherein the obtaining a predicted radiation auxiliary image of a target object at a target radiation time point includes:
obtaining a medical scanning image of the target object at the target radiation time point and an initial treatment plan of the target object, wherein the medical scanning image includes an initial medical scanning image of the target object used to make the initial treatment plan or a medical scanning image of the target object obtained before the target radiation time point; and
obtaining the predicted radiation auxiliary image of the target object at the target radiation time point based on the medical scanning image and the initial treatment plan using a preset conversion algorithm.

19. A system for dose verification, comprising:
at least one storage device storing a set of instructions; and
at least one processor in communication with the storage device, wherein when executing the set of instructions, the at least one processor is configured to cause the system to perform operations including:
obtaining a predicted radiation auxiliary image of a target object at a target radiation time point;
determining a target dose strategy based on the predicted radiation auxiliary image;
performing, based on the target dose strategy, treatment in a current radiation fraction for the target object;
obtaining a radiation auxiliary image of the target object at the target radiation time point; and
reconstructing, based on the radiation auxiliary image, a radiation dose at the target radiation time point.

20. A method for online radiation dose reconstruction implemented on a computing device having one or more processors and one or more storage devices, comprising:
obtaining in real-time a radiation auxiliary image corresponding to each current radiation field in a plurality of radiation fields in a current treatment process for a target object;
reconstructing a radiation dose in real-time based on the radiation auxiliary image corresponding to the current radiation field; and
displaying in real-time the radiation dose corresponding to the current radiation field in the current treatment process, or displaying in real-time a cumulative result of radiation doses corresponding to the plurality of radiation fields in the current treatment process.
